(19) United States
(12) Patent Application Publication
(10) Pub. No.: US 2023/0278968 A1
(43) Pub. Date: Sep. 7, 2023

(54) OXAZINE DYES AND THEIR USE IN NUCLEIC ACID AMPLIFICATION REACTIONS

(71) Applicant: Promega Corporation, Madison, WI (US)

(72) Inventors: Ce Shi, Madison, WI (US); Jian Cao, Madison, WI (US); Wenhui Zhou, Madison, WI (US)

(21) Appl. No.: 18/169,326

(22) Filed: Feb. 15, 2023

Related U.S. Application Data
(60) Provisional application No. 63/310,796, filed on Feb. 16, 2022.

(51) Int. Cl. C07D 265/38; C12Q 1/686; C12Q 1/6876

(52) U.S. Cl. CPC C07D 265/38; C12Q 1/686; C12Q 1/6876

(57) ABSTRACT

Disclosed herein are functionalized oxazine dye compounds, compositions comprising the compounds, and methods of using the compounds, e.g., in nucleic acid amplification reactions. Also disclosed herein are labeled oligonucleotides and labeled nucleotide triphosphate compounds.

### CROSS-REFERENCE TO RELATED APPLICATIONS

This application claims priority to and the benefit of U.S. Provisional Patent Application No. 63/310,796, filed on Feb. 16, 2022, which is incorporated herein by reference in its entirety.

### TECHNICAL FIELD

Disclosed herein are functionalized oxazine dye compounds, compositions comprising the compounds, and methods of using the compounds, e.g., in nucleic acid amplification reactions. Also disclosed herein are labeled oligonucleotides and labeled nucleotide triphosphate compounds.

### BACKGROUND

Fluorescent dyes are widely used in biological research and medical diagnostics. The availability of a wide variety of fluorescent dyes with distinguishable color ranges has made it more practical to perform multiplexed assays, capable of detecting multiple biologic targets at the same time. For particular applications, such as those that involve polymerase chain reaction (PCR), the dyes must be compatible with the reaction conditions, including high temperatures used in the denaturing step. PCR-compatible dyes, particularly those with longer emission wavelengths, are needed.

### SUMMARY

In one aspect, disclosed herein is a compound of formula (I):

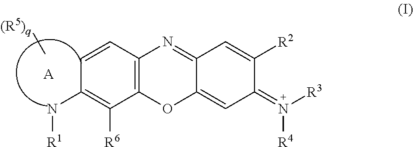

or tautomer or a salt thereof, wherein:

A is a five-, six-, or seven-membered heterocyclyl;

R¹ is selected from hydrogen, C₁-C₆ alkyl, C₁-C₆ heteroalkyl, and -L¹-X, wherein L¹ is alkylene or heteroalkylene, and X is selected from —COOH, —SO₃H, —PO₃H₂, —OPO₃H₂, and a reactive moiety; or R¹ is taken together with R⁶ and the atoms to which they are attached to form a five-, six-, or seven-membered heterocyclyl;

R², R³, and R⁴ are defined follows:

(i) R² is C₁-C₄ alkyl; R³ is hydrogen; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a reactive moiety; or

(ii) R² and R³, together with the atoms to which they are attached, form a five-, six-, or seven-membered heterocyclyl; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a reactive moiety; or

(iii) R² is C₁-C₄ alkyl; and R³ and R⁴, together with the nitrogen atom to which they are attached, form a four-, five-, six-, or seven-membered heterocyclyl;

q is 0, 1, 2, or 3; and

each R⁵ is independently selected from C₁-C₄ alkyl; and

R⁶ is hydrogen, or R⁶ is taken together with R¹ and the atoms to which they are attached to form a five-, six-, or seven-membered heterocyclyl;

wherein:
(a) R¹ is -L¹-X, and X is a reactive moiety selected from an active ester, —N₃, —C≡CH, —N═C═O, —N═C═S, maleimido, —C(O)—CH═CH₂, optionally substituted 1,2,4,5-tetrazinyl, cycloalkenyl, and cycloalkynyl; or
(b) R⁴ is -L²-Z, and Z is a reactive moiety selected from an active ester, —N₃, —C≡CH, —N═C═O, —N═C═S, maleimido, —C(O)—CH═CH₂, optionally substituted 1,2,4,5-tetrazinyl, cycloalkenyl, and cycloalkynyl;

and the compound does not have more than one reactive moiety; wherein the compound is not:

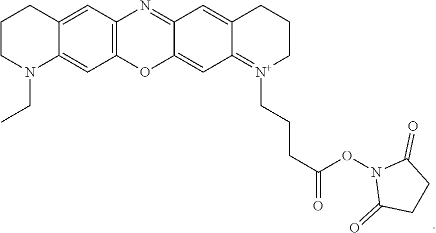 [SMILES: CCN1CCCc2cc3c(cc21)Oc1cc2c(cc1=N3)CCC[N+]=2CCCC(=O)ON1C(=O)CCC1=O]

In some embodiments, A is selected from a pyrrolidine, piperidine, and morpholine ring.

In some embodiments, R¹ is hydrogen, C₁-C₆ alkyl, or C₁-C₆ heteroalkyl. In some embodiments, R¹ is -L¹-X. In some embodiments, L¹ is C₂-C₄ alkylene. In some embodiments, X is selected from —COOH, —SO₃H, —PO₃H₂, and a reactive moiety, wherein the reactive moiety is selected from an active ester, —N₃, —C≡CH, —N═C═O, —N═C═S, maleimido, —C(O)—CH═CH₂, tetrazinyl, cycloalkenyl, and cycloalkynyl.

In some embodiments, R² is C₁-C₄ alkyl; R³ is hydrogen; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a reactive moiety.

In some embodiments, R² and R³, together with the atoms to which they are attached, form a five-, six-, or seven-membered heterocyclyl; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a reactive moiety.

In some embodiments, R⁴ is hydrogen, C₁-C₆ alkyl, or C₁-C₆ heteroalkyl. In some embodiments, R⁴ is -L²-Z. In some embodiments, L² is C₂-C₄ alkylene. In some embodiments, Z is selected from —COOH, —SO₃H, and a reactive moiety, wherein the reactive moiety is selected from an active ester, —N₃, —C≡CH, —N═C═O, —N═C═S, maleimido, —C(O)—CH═CH₂, optionally substituted 1,2,4,5-tetrazinyl, cycloalkenyl, and cycloalkynyl.

In some embodiments, R² is C₁-C₄ alkyl; and R³ and R⁴, together with the nitrogen atom to which they are attached, form a four-, five-, six-, or seven-membered heterocyclyl.

In some embodiments, q is 0.

In some embodiments, R⁶ is hydrogen.

In some embodiments:

A is a pyrrolidine ring;

R¹ is -L¹-X, wherein L¹ is C₂-C₄ alkylene and X is an N-succinimidyl ester reactive moiety;

R² and R³, together with the atoms to which they are attached, form a five-membered heterocyclyl having one nitrogen atom;

R⁴ is selected from C₁-C₆ alkyl and -L²-Z, wherein L² is C₂-C₄ alkylene and Z is —SO₃H;

q is 0; and

R⁶ is hydrogen.

In some embodiments:

A is a morpholine ring;

R¹ is -L¹-X, wherein L¹ is C₂-C₄ alkylene and X is an N-succinimidyl ester reactive moiety;

R² is C₁-C₄ alkyl and R³ is hydrogen; or R² and R³, together with the atoms to which they are attached, form a six-membered ring having one nitrogen atom and one oxygen atom;

R⁴ is selected from C₁-C₆ alkyl and -L²-Z, wherein L² is C₂-C₄ alkylene and Z is —SO₃H;

q is 0; and

R⁶ is hydrogen.

In some embodiments, the compound is selected from:

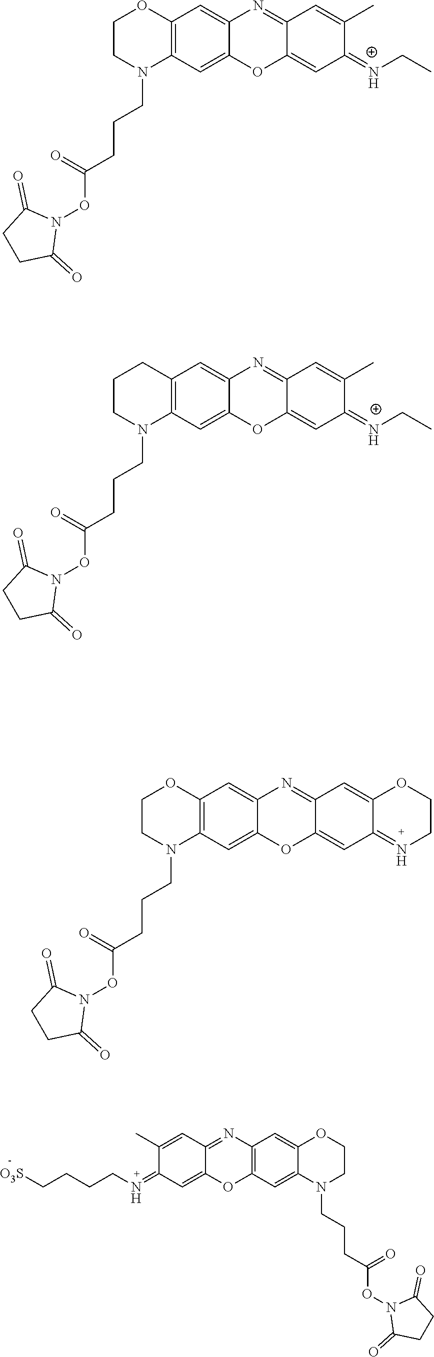

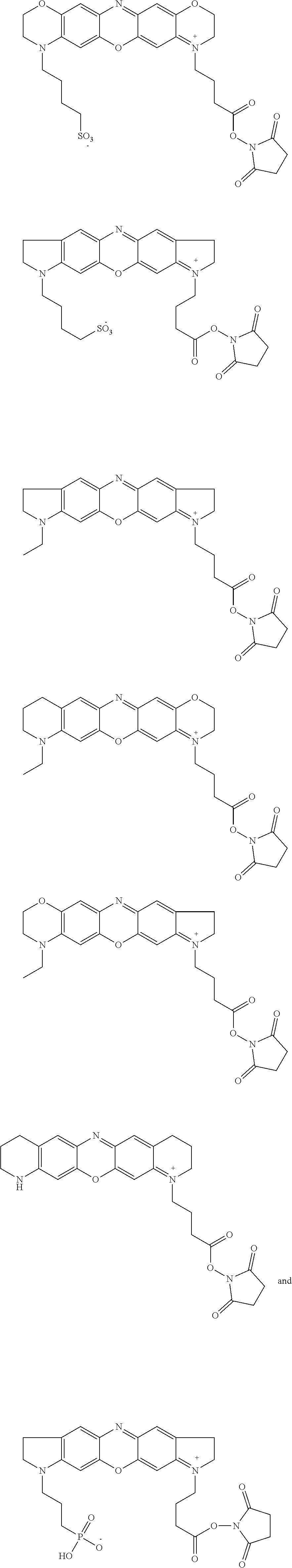

or a tautomer thereof, or a salt thereof.

In another aspect, disclosed herein is an oligonucleotide comprising a moiety of formula (Ia):

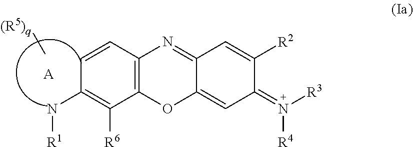

or tautomer or a salt thereof, wherein:

A is a five-, six-, or seven-membered heterocyclyl;

R¹ is selected from hydrogen, C₁-C₆ alkyl, C₁-C₆ heteroalkyl, and -L¹-X, wherein L¹ is alkylene or heteroalkylene, and X is selected from —COOH, —SO₃H, —PO₃H₂, —OPO₃H₂, and a point of attachment to the oligonucleotide; or R¹ is taken together with R⁶ and the atoms to which they are attached to form a five-, six-, or seven-membered heterocyclyl;

R², R³, and R⁴ are defined follows:

(i) R² is C₁-C₄ alkyl; R³ is hydrogen; and R⁴ is selected from hydrogen, C₁-C₆ alkyl, C₁—C₆ heteroalkyl, and -L²-Z, wherein L² is alkylene or heteroalkylene, and Z is selected from —COOH, —SO₃H, —PO₃H₂, —OPO₃H₂, and a point of attachment to the oligonucleotide; or

(ii) R² and R³, together with the atoms to which they are attached, form a five-, six-, or seven-membered heterocyclyl; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a point of attachment to the oligonucleotide; or

(iii) R² is C₁-C₄ alkyl; and R³ and R⁴, together with the nitrogen atom to which they are attached, form a four-, five-, six-, or seven-membered heterocyclyl;

q is 0, 1, 2, or 3; and

each R⁵ is independently selected from C₁-C₄ alkyl; and

R⁶ is hydrogen, or R⁶ is taken together with R¹ and the atoms to which they are attached to form a five-, six-, or seven-membered heterocyclyl;

wherein:
(a) R¹ is -L¹-X, and X is a point of attachment to the oligonucleotide; or
(b) R⁴ is -L²-Z, and Z is a point of attachment to the oligonucleotide
and the moiety of formula (Ia) does not have more than one point of attachment to the oligonucleotide.

In some embodiments, A is selected from a pyrrolidine, piperidine, and morpholine ring.

In some embodiments, R¹ is hydrogen, C₁-C₆ alkyl, or C₁-C₆ heteroalkyl. In some embodiments, R¹ is -L¹-X. In some embodiments, L¹ is C₂-C₄ alkylene. In some embodiments, X is selected from —COOH, —SO₃H, —PO₃H₂, and a point of attachment to the oligonucleotide.

In some embodiments, R² is C₁-C₄ alkyl; R³ is hydrogen; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a point of attachment to the oligonucleotide.

In some embodiments, R² and R³, together with the atoms to which they are attached, form a five-, six-, or seven-membered heterocyclyl; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a point of attachment to the oligonucleotide.

In some embodiments, R⁴ is hydrogen, C₁-C₆ alkyl, or C₁-C₆ heteroalkyl. In some embodiments, R⁴ is -L²-Z. In some embodiments, L² is C₂-C₄ alkylene. In some embodiments, Z is selected from —COOH, —SO₃H, —PO₃H₂, and a point of attachment to the oligonucleotide.

In some embodiments, R² is C₁-C₄ alkyl; and R³ and R⁴, together with the nitrogen atom to which they are attached, form a four-, five-, six-, or seven-membered heterocyclyl.

In some embodiments, q is 0.

In some embodiments, R⁶ is hydrogen.

In some embodiments: A is a pyrrolidine ring; R¹ is -L¹-X, wherein L¹ is C₂-C₄ alkylene and X is a point of attachment to the oligonucleotide; R² and R³, together with the atoms to which they are attached, form a five-membered heterocyclyl having one nitrogen atom; R⁴ is selected from C₁-C₆ alkyl and -L²-Z, wherein L² is C₂-C₄ alkylene and Z is —SO₃H; q is 0; and R⁶ is hydrogen.

In some embodiments: A is a morpholine ring; R¹ is -L¹-X, wherein L¹ is C₂-C₄ alkylene and X is a point of attachment to the oligonucleotide; R² is C₁-C₄ alkyl and R³ is hydrogen; or R² and R³, together with the atoms to which they are attached, form a six-membered ring having one nitrogen atom and one oxygen atom; R⁴ is selected from C₁-C₆ alkyl and -L²-Z, wherein L² is C₂-C₄ alkylene and Z is —SO₃H; q is 0; and R⁶ is hydrogen.

In some embodiments, the oligonucleotide comprises a moiety of formula:

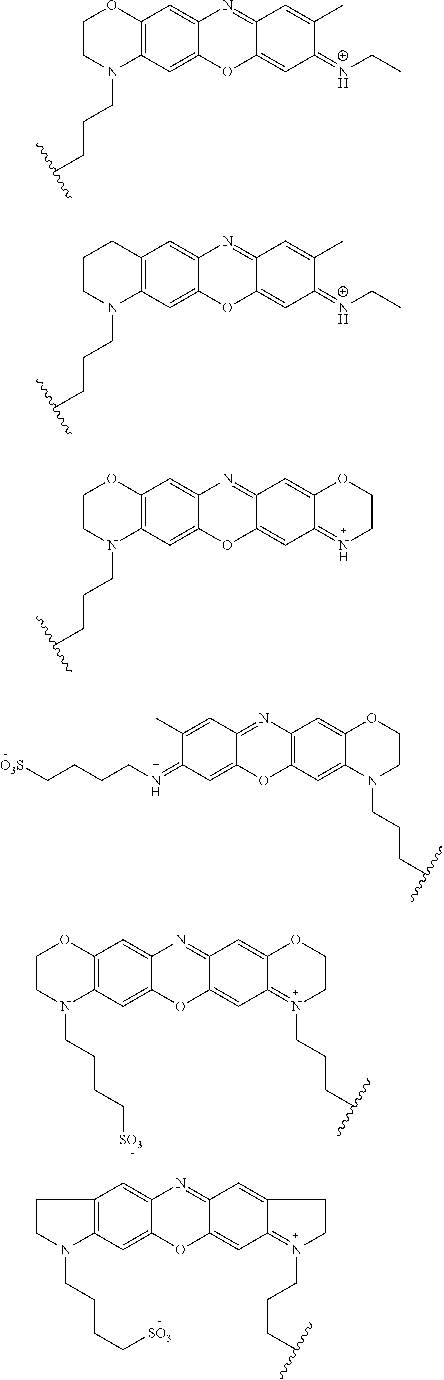

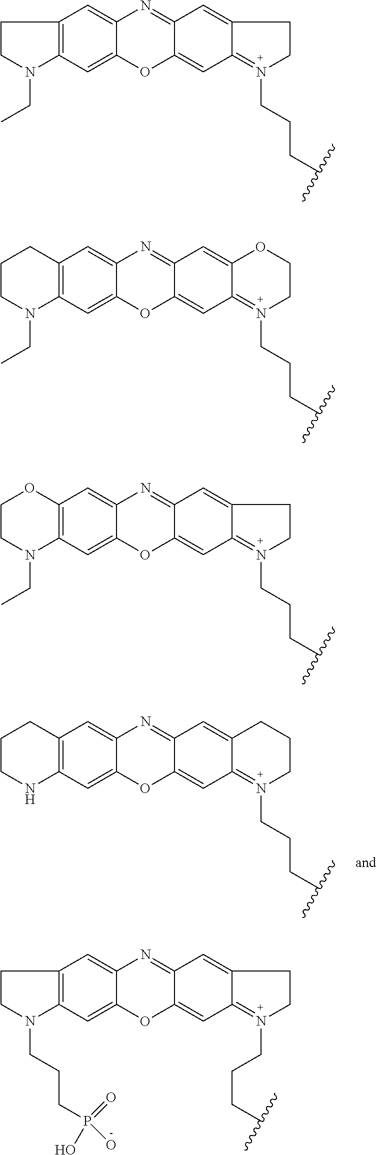

or a tautomer or a salt thereof,
wherein [symbol] represents the point of attachment of the moiety to the oligonucleotide.

In some embodiments, the moiety of formula (Ia) is attached to the oligonucleotide via a direct bond. In some embodiments, the moiety of formula (Ia) is attached to the oligonucleotide via a linker. In some embodiments, the moiety of formula (Ia) is attached to the oligonucleotide via a linker comprising an amide moiety, a carbamate moiety, a five-membered heteroaryl ring, an alkylene moiety, a fused bicyclic heterocycle, or any combination thereof.

In some embodiments, the oligonucleotide is about 5 bases to about 50 bases in length. In some embodiments, the oligonucleotide is about 15 bases to about 35 bases in length.

In another aspect, disclosed herein is a modified nucleotide triphosphate compound comprising a group of formula (Ib):

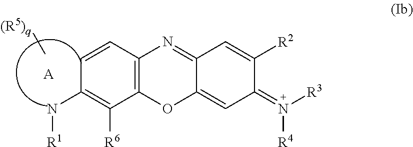

or tautomer or a salt thereof, wherein:

A is a five-, six-, or seven-membered heterocyclyl;

R¹ is selected from hydrogen, C₁-C₆ alkyl, C₁-C₆ heteroalkyl, and -L¹-X, wherein L¹ is alkylene or heteroalkylene, and X is selected from —COOH, —SO₃H, —PO₃H₂, —OPO₃H₂, and a point of attachment to the nucleotide triphosphate compound; or R¹ is taken together with R⁶ and the atoms to which they are attached to form a five-, six-, or seven-membered here;

R², R³, and R⁴ are defined follows:

(i) R² is C₁-C₄ alkyl; R³ is hydrogen; and R⁴ is selected from hydrogen, C₁-C₆ alkyl, C₁—C₆ heteroalkyl, and -L²-Z, wherein L² is alkylene or heteroalkylene, and Z is selected from —COOH, —SO₃H, —PO₃H₂, —OPO₃H₂, and a point of attachment to the nucleotide triphosphate compound; or

(ii) R² and R³, together with the atoms to which they are attached, form a five-, six-, or seven-membered heterocyclyl; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a point of attachment to the nucleotide triphosphate compound; or

(iii) R² is C₁-C₄ alkyl; and R³ and R⁴, together with the nitrogen atom to which they are attached, form a four-, five-, six-, or seven-membered heterocyclyl;

q is 0, 1, 2, or 3; and

each R¹ is independently selected from C₁-C₄ alkyl; and

R⁶ is hydrogen, or R⁶ is taken together with R¹ and the atoms to which they are attached to form a five-, six-, or seven-membered heterocyclyl;

wherein:
(a) R¹ is -L¹-X, and X is a point of attachment to the nucleotide triphosphate compound; or
(b) R⁴ is -L²-Z, and Z is a point of attachment to the nucleotide triphosphate compound,
and the moiety of formula (Ib) does not have more than one point of attachment to the nucleotide triphosphate compound.

In some embodiments, A is selected from a pyrrolidine, piperidine, and morpholine ring.

In some embodiments, R¹ is hydrogen, C₁-C₆ alkyl, or C₁-C₆ heteroalkyl. In some embodiments, R¹ is -L¹-X. In some embodiments, in L¹ is C₂-C₄ alkylene. In some embodiments, X is selected from —COOH, —SO₃H, —PO₃H₂, and a point of attachment to the nucleotide triphosphate compound.

In some embodiments, R² is C₁-C₄ alkyl; R³ is hydrogen; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a point of attachment to the nucleotide triphosphate compound. In some embodiments, R² and R³, together with the atoms to which they are attached, form a five-, six-, or seven-membered heterocyclyl; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a point of attachment to the nucleotide triphosphate compound.

In some embodiments, R⁴ is hydrogen, C₁-C₆ alkyl, or C₁-C₆ heteroalkyl. In some embodiments, R⁴ is -L²-Z. In some embodiments, L² is C₂-C₄ alkylene. In some embodiments, Z is selected from —COOH, —SO₃H, —PO₃H₂, and a point of attachment to the nucleotide triphosphate compound.

In some embodiments, R² is C₁-C₄ alkyl; and R³ and R⁴, together with the nitrogen atom to which they are attached, form a four-, five-, six-, or seven-membered heterocyclyl.

In some embodiments, q is 0.

In some embodiments, R⁶ is hydrogen.

In some embodiments: A is a pyrrolidine ring; R¹ is -L¹-X, wherein L¹ is C₂-C₄ alkylene and X is a point of attachment to the nucleotide triphosphate compound; R² and R³, together with the atoms to which they are attached, form a five-membered heterocyclyl having one nitrogen atom; R⁴ is selected from C₁-C₆ alkyl and -L²-Z, wherein L² is C₂-C₄ alkylene and Z is —SO₃H; q is 0; and R⁶ is hydrogen.

In some embodiments: A is a morpholine ring; R¹ is -L¹-X, wherein L¹ is C₂-C₄ alkylene and X is a point of attachment to the nucleotide triphosphate compound; R² is C₁-C₄ alkyl and R³ is hydrogen; or R² and R³, together with the atoms to which they are attached, form a six-membered ring having one nitrogen atom and one oxygen atom; R⁴ is selected from C₁-C₆ alkyl and L²-Z, wherein L² is C₂-C₄ alkylene and Z is —SO₃H; q is 0; and R⁶ is hydrogen.

In some embodiments, the nucleotide triphosphate compound comprises a moiety of formula:

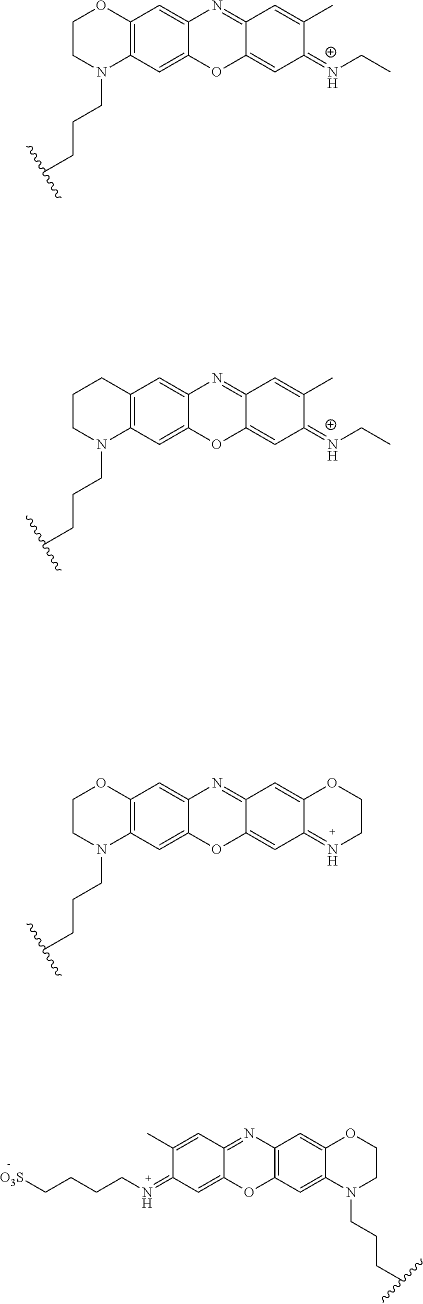

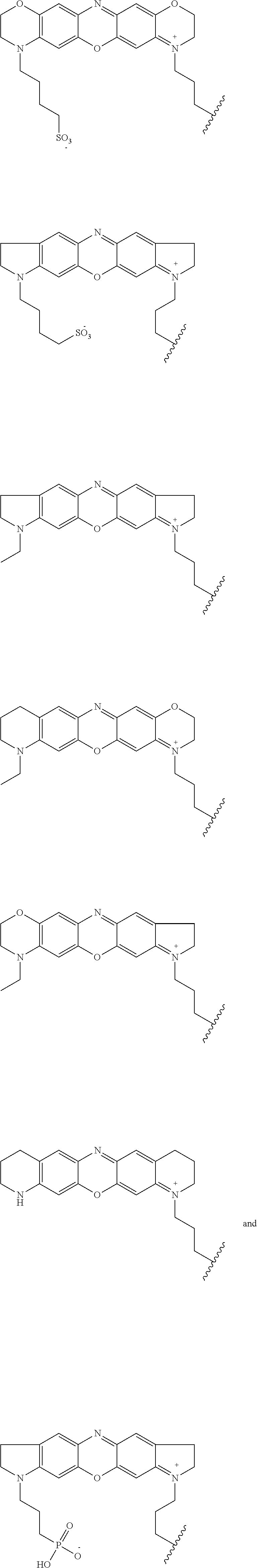

or a tautomer or a salt thereof,

wherein [symbol] represents the point of attachment of the moiety to the nucleotide triphosphate compound.

In some embodiments, the moiety of formula (Ib) is attached to the nucleotide triphosphate compound via a direct bond. In some embodiments, the moiety of formula (Ib) is attached to the nucleotide triphosphate compound via a linker. In some embodiments, the moiety of formula (Ib) is attached to the nucleotide triphosphate compound via a linker comprising an amide moiety, a carbamate moiety, a five-membered heteroaryl ring, an alkylene moiety, a fused bicyclic heterocycle, or any combination thereof.

In some embodiments, the compound is a modified deoxynucleotide triphosphate compound selected from deoxyadenosine triphosphate, deoxycytidine triphosphate, deoxyguanosine triphosphate, and deoxythymidine triphosphate. In some embodiments, the compound is a modified dideoxynucleotide triphosphate compound selected from dideoxyadenosine triphosphate, dideoxycytidine triphosphate, dideoxyguanosine triphosphate, and dideoxythymidine triphosphate.

In another aspect, disclosed herein is a method of performing a nucleic acid amplification reaction, comprising:
(a) adding an oligonucleotide compound disclosed herein (e.g., an oligonucleotide comprising a moiety of formula (Ia)) to a reaction mixture; and
(b) performing the amplification reaction.

In some embodiments, the nucleic acid amplification reaction is selected from the group consisting of: polymerase chain reaction (PCR), quantitative PCR, real time PCR, hot start PCR, single cell PCR, nested PCR, in situ colony PCR, digital PCR (dPCR), Droplet Digital™ PCR (ddPCR), emulsion PCR, ligase chain reaction (LCR), transcription based amplification system (TAS), nucleic acid sequence-based amplification (NASBA), strand displacement amplification (SDA), rolling circle amplification (RCA), and hyper-branched RCA (HRCA). In some embodiments, the nucleic acid amplification reaction is a multiplex nucleic acid amplification reaction.

In another aspect, disclosed herein is a method of performing a chain termination DNA sequencing reaction, the method comprising:
(a) adding a modified dideoxynucleotide triphosphate compound disclosed herein (e.g., a modified dideoxynucleotide triphosphate compound comprising a moiety of formula (Ib)) to a polymerase chain reaction (PCR) mixture and performing PCR;
(b) removing unincorporated modified dideoxynucleotide triphosphate compounds from the PCR mixture; and
(c) performing sequencing analysis.

In some embodiments, the sequencing analysis comprises fragment analysis and/or Sanger sequencing analysis.

In another aspect, disclosed herein is a method of performing a chain termination DNA sequencing reaction, the method comprising:
(a) adding a modified deoxynucleotide triphosphate compound disclosed herein (e.g., a modified dideoxynucleotide triphosphate compound comprising a moiety of formula (Ib)) to a polymerase chain reaction (PCR) mixture, and
(b) performing PCR,

wherein a fluorescent signal from the PCR mixture indicates which dNTP has been added, and wherein a terminator is cleaved to facilitate addition of a subsequent dNTP.

In some embodiments, the method further comprises performing fragment analysis and/or next generation sequencing. In some embodiments, the method is multiplexed.

### BRIEF DESCRIPTION OF THE DRAWINGS

FIG. 1 shows electropherograms of amplified samples using oligonucleotide primers labeled with dye compounds disclosed herein (JC-0025 and JC-0081).

FIG. 2 shows electropherograms of amplified samples using oligonucleotide primers labeled with dye compounds disclosed herein (CS-1341, CS-1377, and JC-0084).

### DETAILED DESCRIPTION

Disclosed herein are oxazine dye compounds that are compatible with PCR reaction conditions. The dyes include a reactive moiety that can be used, for example, to label oligonucleotide primers and nucleotide triphosphate compounds (dNTPs). The dyes, or compounds labeled with the dyes (e.g., labeled oligonucleotide primers and labeled dNTPs, can be used in a variety of sequencing methods, including multiplex PCR assays.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; and Carruthers, Some Modern Methods of Organic Synthesis, 3^{rd} Edition, Cambridge University Press, Cambridge, 1987; the entire contents of each of which are incorporated herein by reference.

As used herein, the term “alkyl” means a straight or branched, saturated hydrocarbon chain. An alkyl group can have, for example, 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), 1 to 4 carbon atoms (C₁-C₄ alkyl), 6 to 20 carbon atoms (C₆-C₂₀ alkyl), or 8 to 14 carbon atoms (C₈-C₁₄ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “alkylene” refers to a divalent group derived from a straight or branched, saturated hydrocarbon chain. Representative examples of alkylene include, but are not limited to, —CH₂—, —CH₂CH₂—, —CH(CH₃)—, —CH₂CH₂CH₂—, —CH₂CH(CH₃)—, —CH₂CH₂CH₂CH₂—, —CH₂CH(CH₃)CH₂—, —CH₂CH₂CH(CH₃)—, —CH₂CH₂CH₂CH₂CH₂—, —CH₂CH(CH₃)CH₂CH₂—, —CH(CH₃)CH₂CH₂CH₂—, —CH₂CH₂CH₂CH₂CH₂CH₂—, —CH₂CH₂CH(CH₃)CH₂CH₂—, —CH₂CH(CH₃)CH₂CH₂CH₂—, and —CH(CH₃)CH₂CH₂CH₂CH₂—.

As used herein, the term “active ester” refers to an ester functional group that is highly susceptible toward nucleophilic attack, e.g., by a functional group such as an amine or a thiol. Examples include, but are not limited to, N-hydroxysuccinimidyl esters, N-hydroxysulfosuccinimidyl esters, pentafluorophenyl esters, and the like.

As used herein, the term “cycloalkyl” refers to a saturated carbocyclic ring system containing three to ten carbon atoms and zero heteroatoms. The cycloalkyl may be monocyclic, bicyclic, bridged, fused, or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, bicyclo[2.2.1]heptanyl, bicyclo[3.2.1]octanyl, and bicyclo[5.2.0]nonanyl.

As used herein, the term “cycloalkenyl” refers to a non-aromatic, monocyclic or multicyclic, carbocyclic ring system containing at least one carbon-carbon double bond. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl). Exemplary monocyclic cycloalkenyl rings include cyclopentenyl, cyclohexenyl, and cycloheptenyl. Exemplary monocyclic cycloalkenyl rings include cyclopentenyl, cyclohexenyl, and cycloheptenyl.

As used herein, the term “cycloalkynyl” refers to a non-aromatic, monocyclic or multicyclic, carbocyclic ring system containing at least one carbon-carbon triple bond. Examples of cycloalkynyl groups include, but are not limited to, cycloheptynyl, cyclooctynyl, cyclononynyl, and the like.

As used herein, the term “heteroalkyl” means an alkyl group, as defined herein, in which one or more of the carbon atoms (and any associated hydrogen atoms) are each independently replaced with a heteroatom group such as —NH—, —O—, —S—, —S(O)—, —S(O)₂—, and the like. By way of example, 1, 2, or 3 carbon atoms may be independently replaced with the same or different heteroatomic group. Examples of heteroalkyl groups include, but are not limited to, —OCH₃, —CH₂OCH₃, —SCH₃, —CH₂SCH₃, —NHCH₃, and —CH₂NHCH₃, where R is hydrogen, alkyl, aryl, arylalkyl, heteroalkyl, or heteroaryl, each of which may be optionally substituted. Heteroalkyl also includes groups in which a carbon atom of the alkyl is oxidized (i.e., is —C(O)—).

As used herein, the term “heteroalkylene” means an alkylene group, as defined herein, in which one or more of the carbon atoms (and any associated hydrogen atoms) are each independently replaced with a heteroatom group such as —NH—, —O—, —S—, —S(O)—, —S(O)₂—, and the like. By way of example, 1, 2, or 3 carbon atoms may be independently replaced with the same or different heteroatomic group. Heteroalkylene also includes groups in which a carbon atom of the alkylene is oxidized (i.e., is —C(O)—). Examples of heteroalkylene groups include, but are not limited to, —CH₂—O—CH₂—, —CH₂—S—CH₂—, —CH₂—NH—CH₂—, —CH₂—NH—C(O)—CH₂—, and the like, as well as polyethylene oxide chains, polypropylene oxide chains, and polyethyleneimine chains.

As used herein, the term “heterocycle” or “heterocyclic” refers to a saturated or partially unsaturated non-aromatic cyclic group having one or more ring heteroatoms independently selected from O, N, and S. means a monocyclic heterocycle, a bicyclic heterocycle, or a tricyclic heterocycle. The monocyclic heterocycle is a three-, four-, five-, six-, seven-, or eight-membered ring containing at least one heteroatom independently selected from O, N, and S. The three- or four-membered ring contains zero or one double bond, and one heteroatom selected from O, N, and S. The five-membered ring contains zero or one double bond and one, two or three heteroatoms selected from O, N, and S. The six-membered ring contains zero, one, or two double bonds and one, two, or three heteroatoms selected from O, N, and S. The seven- and eight-membered rings contains zero, one, two, or three double bonds and one, two, or three heteroatoms selected from O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, 2-azaspiro[3.3]heptan-2-yl, azabicyclo[2.2.1]heptyl (including 2-azabicyclo[2.2.1]hept-2-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan,hexahydro-1H-1,4-methanocyclopenta[c]furan, aza-adamantane (1-azatricyclo[3.3.1.^{3,7}]decane), and oxa-adamantane (2-oxatricyclo[3.3.1^{3,7}]decane). The monocyclic, bicyclic, and tricyclic heterocycles are connected to the parent molecular moiety through any carbon atom or any nitrogen atom contained within the rings.

### Compounds

Disclosed herein are compounds of formula (I):

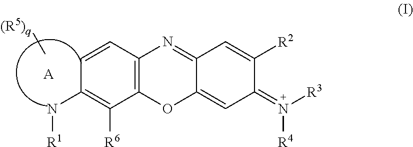

or tautomer or a salt thereof, wherein:

A is a five-, six-, or seven-membered heterocyclyl;

R¹ is selected from hydrogen, C₁-C₆ alkyl, C₁-C₆ heteroalkyl, and -L¹-X, wherein L¹ is alkylene or heteroalkylene, and X is selected from —COOH, —SO₃H, —PO₃H₂, —OPO₃H₂, and a reactive moiety; or R¹ is taken together with R⁶ and the atoms to which they are attached to form a five-, six-, or seven-membered heterocyclyl;

R², R³, and R⁴ are defined follows:

(i) R² is C₁-C₄ alkyl; R³ is hydrogen; and R⁴ is selected from hydrogen, C₁-C₆ alkyl, C₁—C₆ heteroalkyl, and -L²-Z, wherein L² is alkylene or heteroalkylene, and Z is selected from —COOH, —SO₃H, —PO₃H₂, —OPO₃H₂, and a reactive moiety; or

(ii) R² and R³, together with the atoms to which they are attached, form a five-, six-, or seven-membered heterocyclyl; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a reactive moiety; or

(iii) R² is C₁-C₄ alkyl; and R³ and R⁴, together with the nitrogen atom to which they are attached, form a four-, five-, six-, or seven-membered heterocyclyl;

q is 0, 1, 2, or 3; and

each R⁵ is independently selected from C₁-C₄ alkyl; and

R⁶ is hydrogen, or R⁶ is taken together with R¹ and the atoms to which they are attached to form a five-, six-, or seven-membered heterocyclyl;

wherein:
(a) R¹ is and X is a reactive moiety selected from an active ester, —N₃, —C≡CH, —N═C═O, —N═C═S, maleimido, —C(O)—CH═CH₂, optionally substituted 1,2,4,5-tetrazinyl, cycloalkenyl, and cycloalkynyl; or
(b) R⁴ is -L²-Z, and Z is a reactive moiety selected from an active ester, —N₃, —C≡CH, —N═C═O, —N═C═S, maleimido, —C(O)—CH═CH₂, optionally substituted 1,2,4,5-tetrazinyl, cycloalkenyl, and cycloalkynyl;
and the compound does not have more than one reactive moiety; wherein the compound is not:

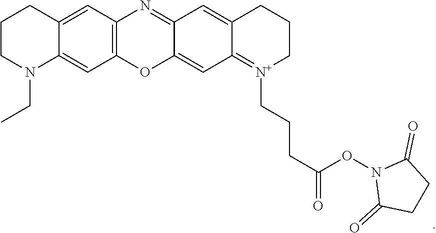 [SMILES: CCN1CCCc2cc3c(cc21)Oc1cc2c(cc1=N3)CCC[N+]=2CCCC(=O)ON1C(=O)CCC1=O]

In some embodiments, A is selected from a pyrrolidine, piperidine, and morpholine ring. In some embodiments, A is a morpholine ring. In some embodiments, A is a piperidine ring. In some embodiments, A is a pyrrolidine ring.

In some embodiments, R¹ is hydrogen, C₁-C₆ alkyl, or C₁-C₆ heteroalkyl. In some embodiments, R¹ is hydrogen or C₁-C₄ alkyl. In some embodiments, R¹ is hydrogen or ethyl. In some embodiments, R¹ is hydrogen. In some embodiments, R¹ is ethyl.

In some embodiments, R¹ is In some embodiments, L¹ is C₂-C₄ alkylene or L¹ is C₂-C₄ heteroalkylene. In some embodiments, L¹ is C₂-C₄ alkylene. In some embodiments, L¹ is —CH₂CH₂CH₂—. In some embodiments, X is selected from —COOH, —SO₃H, —PO₃H₂, and a reactive moiety, wherein the reactive moiety is selected from an active ester, —N₃, —C≡CH, —N═C═O, —N═C═S, maleimido, —C(O)—CH═CH₂, tetrazinyl, cycloalkenyl, and cycloalkynyl. In some embodiments, X is selected from —SO₃H, —PO₃H₂, and an active ester. In some embodiments, X is —SO₃H. In some embodiments, X is —PO₃H₂. In some embodiments, X is an active ester, such as a succinimidyl ester or a pentafluorophenyl ester. In some embodiments, X is a succinimidyl ester, which is either unsubstituted or substituted with a —SO₃H group (or a salt thereof). In some embodiments, X is selected from:

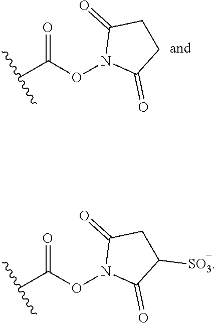

In some embodiments, R² is C₁-C₄ alkyl, R³ is hydrogen, and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a reactive moiety. In some embodiments, R² is C₁-C₄ alkyl, R³ is hydrogen, and R⁴ is selected from C₁-C₆ alkyl and -L²-Z, wherein L² is alkylene or heteroalkylene, and Z is —SO₃H. In some embodiments, R² is C₁-C₂ alkyl, R³ is hydrogen, and R⁴ is C₁-C₂ alkyl or —(CH₂)ₙ—SO₃H, wherein n is 3, 4, or 5. In some embodiments, R² is methyl, R³ is hydrogen, and R⁴ is methyl, ethyl or —(CH₂)₄—SO₃H. In some embodiments, R² is methyl, R³ is hydrogen, and R⁴ is ethyl. In some embodiments, R² is methyl, R³ is hydrogen, and R⁴ is —(CH₂)₄—SO₃H.

In some embodiments, R² and R³, together with the atoms to which they are attached, form a five-, six-, or seven-membered heterocyclyl; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a reactive moiety. In some embodiments, R² and R³, together with the atoms to which they are attached, form a five-, six-, or seven-membered heterocyclyl; and R⁴ is selected from from hydrogen, C₁-C₆ alkyl, and C₁-C₆ heteroalkyl. In some embodiments, R² and R³, together with the atoms to which they are attached, form a five-, six-, or seven-membered heterocyclyl; and R⁴ is selected from hydrogen, and C₁-C₂ alkyl. In some embodiments, R² and R³, together with the atoms to which they are attached, form a five-, six-, or seven-membered heterocyclyl; and R⁴ is -L²-Z. In some embodiments, L² is C₂-C₄ alkylene. In some embodiments, L² is —CH₂CH₂CH₂—. In some embodiments, Z is selected from —COOH, —SO₃H, and a reactive moiety, wherein the reactive moiety is selected from an active ester, —N₃, —C≡CH, —N═C═O, —N═C═S, maleimido, —C(O)—CH═CH₂, optionally substituted 1,2,4,5-tetrazinyl, cycloalkenyl, and cycloalkynyl. In some embodiments, Z is an active ester. In some embodiments, Z is a succinimidyl ester.

In some embodiments, R² and R³, together with the atoms to which they are attached, form a five- or six-membered heterocyclyl; and R⁴ is selected from hydrogen, C₁-C₆ alkyl, C₁-C₆ heteroalkyl, and and -L²-Z, wherein L² is alkylene or heteroalkylene, and Z is selected from —COOH, —SO₃H, —PO₃H₂, —OPO₃H₂, and a reactive moiety. In some embodiments, R² and R³, together with the atoms to which they are attached, form a five- or six-membered heterocyclyl; and R⁴ is selected from hydrogen and and -L²-Z, wherein L² is C₂-C₄ alkylene or C₂-C₄ heteroalkylene, and Z is a reactive moiety selected from an active ester, —N₃, —C≡CH, —N═C═O, —N═C═S, maleimido, —C(O)—CH═CH₂, tetrazinyl, cycloalkenyl, and cycloalkynyl. In some embodiments, R² and R³, together with the atoms to which they are attached, form a five- or six-membered heterocyclyl; and R⁴ is selected from hydrogen and -L²-Z, wherein L² is —CH₂CH₂CH₂—, and Z is a reactive moiety selected from an active ester, such as a succinimidyl ester. In some embodiments, the group

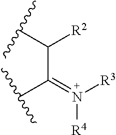

in formula (I) has a structure selected from:

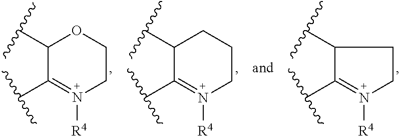

wherein R⁴ is as defined and described above.

In some embodiments, R² is C₁-C₄ alkyl; and R³ and R⁴, together with the nitrogen atom to which they are attached, form a four-, five-, six-, or seven-membered heterocyclyl. In some embodiments, R² is methyl; and R³ and R⁴, together with the nitrogen atom to which they are attached, form a four-, five-, six-, or seven-membered heterocyclyl.

In some embodiments, q is 0. In some embodiments, q is 1 and R⁵ is methyl.

In some embodiments, R⁶ is hydrogen.

In some embodiments, in the compound of formula (I):

A is a pyrrolidine ring;

R¹ is -L¹-X, wherein L¹ is C₂-C₄ alkylene and X is an N-succinimidyl ester reactive moiety;

R² and R³, together with the atoms to which they are attached, form a five-membered heterocyclyl having one nitrogen atom;

R⁴ is selected from C₁-C₆ alkyl and -L²-Z, wherein L² is C₂-C₄ alkylene and Z is —SO₃H;

q is 0; and

R⁶ is hydrogen.

In some embodiments, in the compound of formula (I):

A is a morpholine ring;

R¹ is wherein L¹ is C₂-C₄ alkylene and X is an N-succinimidyl ester reactive moiety;

R² is C₁-C₄ alkyl and R³ is hydrogen; or R² and R³, together with the atoms to which they are attached, form a six-membered ring having one nitrogen atom and one oxygen atom;

R⁴ is selected from C₁-C₆ alkyl and -L²-Z, wherein L² is C₂-C₄ alkylene and Z is —SO₃H;

q is 0; and

R⁶ is hydrogen.

In some embodiments, the compound is selected from:

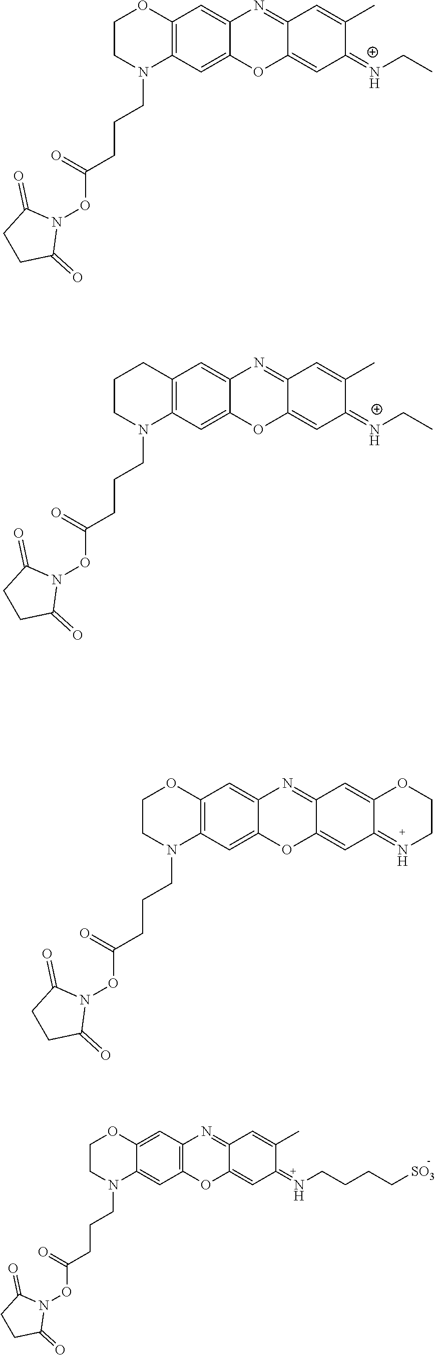

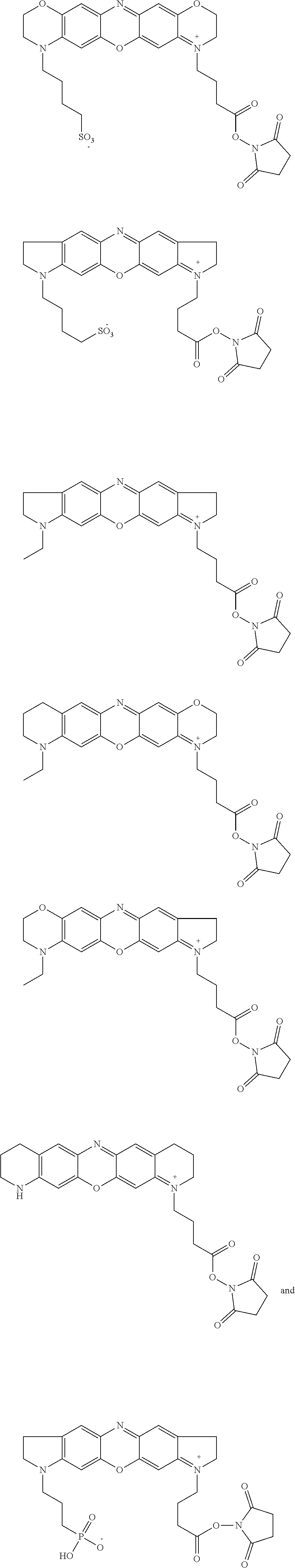

and tautomers and salts thereof.

Additional compounds include the following:

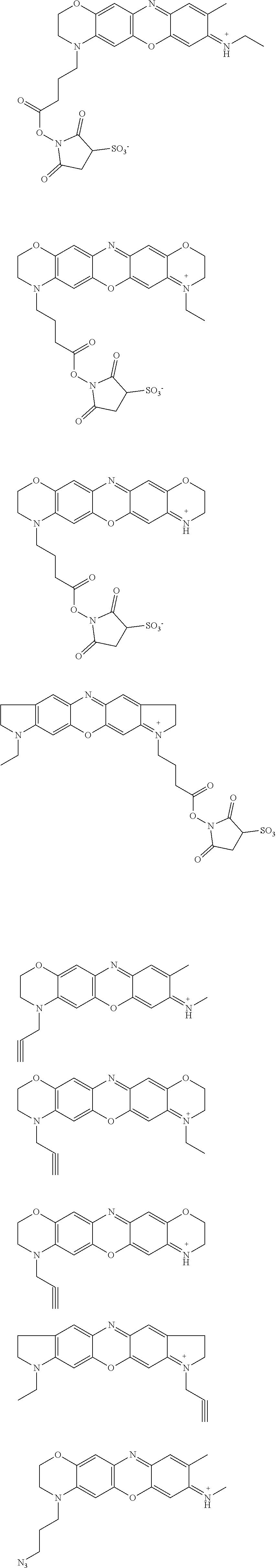

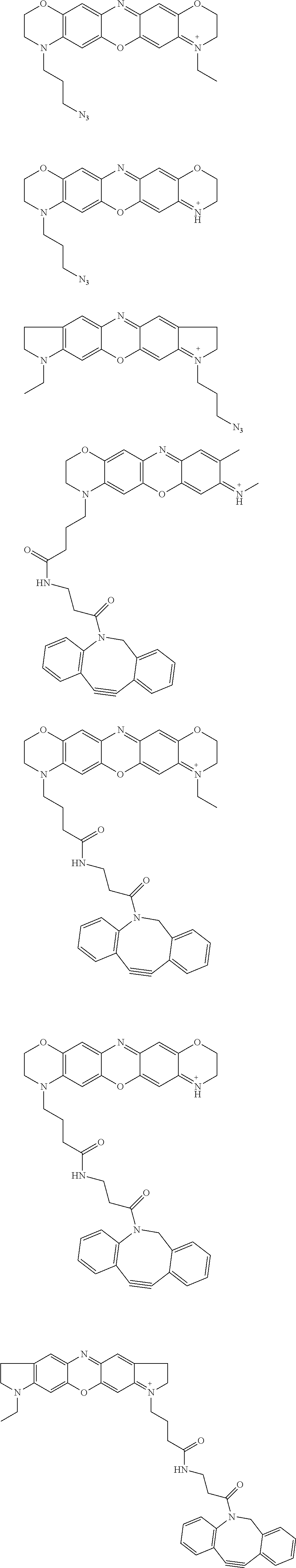

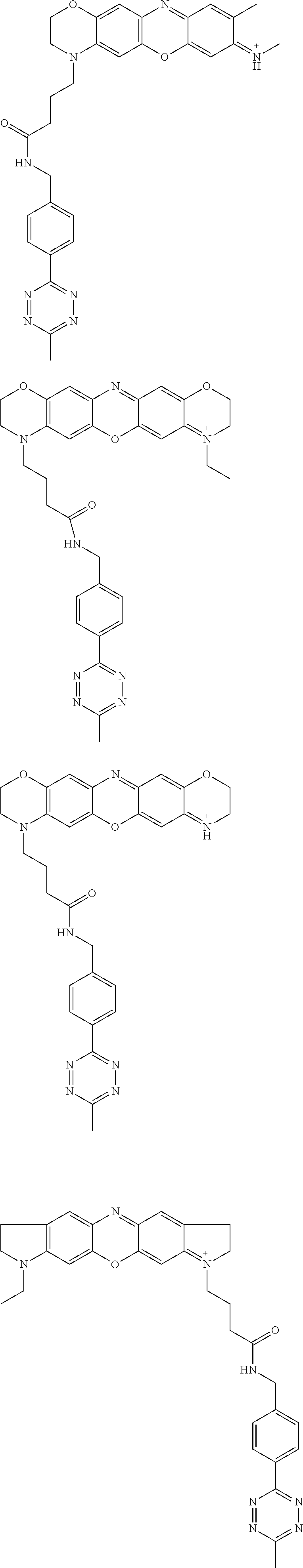

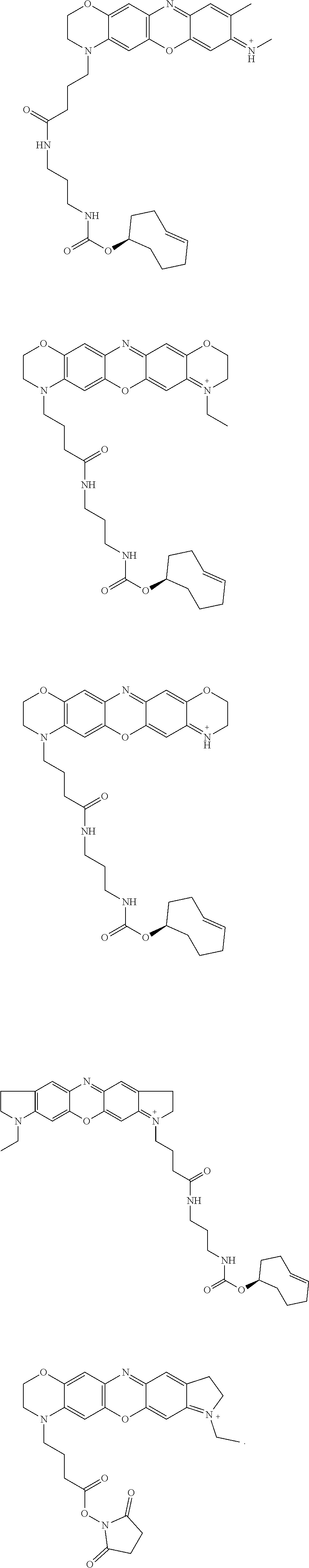

Compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry,” 5^{th} edition (1989), by Furniss, Hannaford, Smith, and Tatchell, pub. Longman Scientific & Technical, Essex CM20 2JE, England.

Reaction conditions and reaction times for each individual step can vary depending on the particular reactants employed and substituents present in the reactants used. Reactions can be worked up in a conventional manner, e.g., by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature.

Standard experimentation, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the disclosure. Suitable protecting groups and the methods for protecting and deprotecting different substituents using such suitable protecting groups are well known to those skilled in the art; examples of which can be found in P G M Wuts and T W Greene, in Greene's book titled Protective Groups in Organic Synthesis (4^{th} ed.), John Wiley & Sons, NY (2006).

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization, or enzymatic resolution).

Similarly, when a pure geometric isomer of a compound is required, it can be obtained by carrying out one of the procedures described herein using a pure geometric isomer as a starting material, or by resolution of a mixture of the geometric isomers of the compound or intermediates using a standard procedure such as chromatographic separation.

The synthetic schemes and specific examples disclosed herein are illustrative and are not to be read as limiting the scope of the disclosure or the claims. Alternatives, modifications, and equivalents of the synthetic methods and specific examples are contemplated.

The compounds illustrated above are either in zwitterionic form or have a net charge, in which case the compounds will further include an ion to balance the net charge. In particular, if the compound is anionic or has a functional group that may be anionic (e.g., —COOH may be —COO⁻), then a salt may be formed with one or more suitable cations. Examples of suitable inorganic cations include, but are not limited to, alkali metal cations such as Li⁺, Na⁺, and K⁺, alkaline earth cations such as Ca²⁺, Mg²⁺, and other cations. Sodium salts may be particularly suitable. Examples of suitable organic cations include, but are not limited to, ammonium ion (i.e., NH₄⁺) and substituted ammonium ions (e.g., NH₃R₁⁺, NH₂R₂⁺, NHR₃⁺, and NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids such as lysine and arginine. In some embodiments, the compound is a sodium salt. If the compound is cationic or has a functional group that may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, tetrafluoroboric, toluenesulfonic, trifluoroacetic, trifluoromethanesulfonic, and valeric. In some embodiments, compounds disclosed herein are trifluoroacetate salts.

The present disclosure also includes isotopically-labeled compounds, which are identical to those disclosed herein but for the fact that one or more atoms are replaced by an atom having an atomic mass or mass number different from the atomic mass or mass number usually found in nature. Examples of isotopes suitable for inclusion in the compounds of the disclosure are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ³¹P, ³⁵S, ¹⁸F, and ³⁶Cl, respectively. Isotopically-labeled compounds of formula (I) or (II) can generally be prepared by conventional techniques known to those skilled in the art or by processes analogous to those described herein using an appropriate isotopically-labeled reagent in place of a non-isotopically-labeled reagent.

### Oligonucleotides

Also disclosed herein are oligonucleotides that are labeled with the dye compounds disclosed herein, such as the dye compounds of formula (I). For example, disclosed herein are oligonucleotides comprising a moiety of formula (Ia):

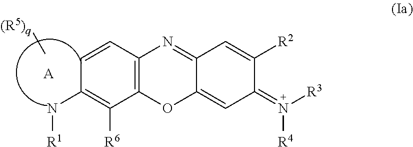

or tautomer or a salt thereof, wherein:

A is a five-, six-, or seven-membered heterocyclyl;

R¹ is selected from hydrogen, C₁-C₆ alkyl, C₁-C₆ heteroalkyl, and -L¹-X, wherein L¹ is alkylene or heteroalkylene, and X is selected from —COOH, —SO₃H, —PO₃H₂, —OPO₃H₂, and a point of attachment to the oligonucleotide; or R¹ is taken together with R⁶ and the atoms to which they are attached to form a five-, six-, or seven-membered heterocyclyl;

R², R³, and R⁴ are defined follows:

(i) R² is C₁-C₄ alkyl; R³ is hydrogen; and R⁴ is selected from hydrogen, C₁-C₆ alkyl, C₁—C₆ heteroalkyl, and -L²-Z, wherein L² is alkylene or heteroalkylene, and Z is selected from —COOH, —SO₃H, —PO₃H₂, —OPO₃H₂, and a point of attachment to the oligonucleotide; or

(ii) R² and R³, together with the atoms to which they are attached, form a five-, six-, or seven-membered heterocyclyl; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a point of attachment to the oligonucleotide; or

(iii) R² is C₁-C₄ alkyl; and R³ and R⁴, together with the nitrogen atom to which they are attached, form a four-, five-, six-, or seven-membered heterocyclyl;

q is 0, 1, 2, or 3; and

each R⁵ is independently selected from C₁-C₄ alkyl; and

R⁶ is hydrogen, or R⁶ is taken together with R¹ and the atoms to which they are attached to form a five-, six-, or seven-membered heterocyclyl;

wherein:
(a) R¹ is -L¹-X, and X is a point of attachment to the oligonucleotide; or
(b) R⁴ is -L²-Z, and Z is a point of attachment to the oligonucleotide

and the moiety of formula (Ia) does not have more than one point of attachment to the oligonucleotide.

In some embodiments, A is selected from a pyrrolidine, piperidine, and morpholine ring. In some embodiments, A is a morpholine ring. In some embodiments, A is a piperidine ring. In some embodiments, A is a pyrrolidine ring.

In some embodiments, R¹ is hydrogen, C₁-C₆ alkyl, or C₁-C₆ heteroalkyl. In some embodiments, R¹ is hydrogen or C₁-C₄ alkyl. In some embodiments, R¹ is hydrogen or ethyl. In some embodiments, R¹ is hydrogen. In some embodiments, R¹ is ethyl.

In some embodiments, R¹ is -L¹-X. In some embodiments, L¹ is C₂-C₄ alkylene or L¹ is C₂-C₄ heteroalkylene. In some embodiments, L¹ is C₂-C₄ alkylene. In some embodiments, L¹ is —CH₂CH₂CH₂—. In some embodiments, X is selected from —COOH, —SO₃H, —PO₃H₂, and a point of attachment to the oligonucleotide. In some embodiments, X is selected from —SO₃H, —PO₃H₂, and a point of attachment to the oligonucleotide. In some embodiments, X is —SO₃H. In some embodiments, X is —PO₃H₂. In some embodiments, X is a point of attachment to the oligonucleotide.

In some embodiments, R² is C₁-C₄ alkyl, R³ is hydrogen, and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a point of attachment to the oligonucleotide. In some embodiments, R² is C₁-C₄ alkyl, R³ is hydrogen, and R⁴ is selected from C₁-C₆ alkyl and -L²-Z, wherein L² is alkylene or heteroalkylene, and Z is —SO₃H. In some embodiments, R² is C₁-C₂ alkyl, R³ is hydrogen, and R⁴ is C₁-C₂ alkyl or —(CH₂)ₙ—SO₃H, wherein n is 3, 4, or 5. In some embodiments, R² is methyl, R³ is hydrogen, and R⁴ is methyl, ethyl or —(CH₂)₄—SO₃H. In some embodiments, R² is methyl, R³ is hydrogen, and R⁴ is ethyl. In some embodiments, R² is methyl, R³ is hydrogen, and R⁴ is —(CH₂)₄—SO₃H.

In some embodiments, R² and R³, together with the atoms to which they are attached, form a five-, six-, or seven-membered heterocyclyl; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a point of attachment to the oligonucleotide. In some embodiments, R² and R³, together with the atoms to which they are attached, form a five-, six-, or seven-membered heterocyclyl; and R⁴ is selected from from hydrogen, C₁-C₆ alkyl, and C₁-C₆ heteroalkyl. In some embodiments, R² and R³, together with the atoms to which they are attached, form a five-, six-, or seven-membered heterocyclyl; and R⁴ is selected from hydrogen, and C₁-C₂ alkyl. In some embodiments, R² and R³, together with the atoms to which they are attached, form a five-, six-, or seven-membered heterocyclyl; and R⁴ is -L²-Z. In some embodiments, L² is C₂-C₄ alkylene. In some embodiments, L² is —CH₂CH₂CH₂—. In some embodiments, Z is selected from —COOH, —SO₃H, and a point of attachment to the oligonucleotide.

In some embodiments, R² and R³, together with the atoms to which they are attached, form a five- or six-membered heterocyclyl; and R⁴ is selected from hydrogen, C₁-C₆ alkyl, C₁-C₆ heteroalkyl, and and -L²-Z, wherein L² is alkylene or heteroalkylene, and Z is selected from —COOH, —SO₃H, —PO₃H₂, —OPO₃H₂, and a point of attachment to the oligonucleotide. In some embodiments, R² and R³, together with the atoms to which they are attached, form a five- or six-membered heterocyclyl; and R⁴ is selected from hydrogen and and -L²-Z, wherein L² is C₂-C₄ alkylene or C₂-C₄ heteroalkylene, and Z is a point of attachment to the oligonucleotide. In some embodiments, R² and R³, together with the atoms to which they are attached, form a five- or six-membered heterocyclyl; and R⁴ is selected from hydrogen and -L²-Z, wherein L² is —CH₂CH₂CH₂—, and Z is a point of attachment to the oligonucleotide.

In some embodiments, R² is C₁-C₄ alkyl; and R³ and R⁴, together with the nitrogen atom to which they are attached, form a four-, five-, six-, or seven-membered heterocyclyl. In some embodiments, R² is methyl; and R³ and R⁴, together with the nitrogen atom to which they are attached, form a four-, five-, six-, or seven-membered heterocyclyl.

In some embodiments, q is 0. In some embodiments, q is 1 and R⁵ is methyl.

In some embodiments, R⁶ is hydrogen.

In some embodiments, in the moiety of formula (Ia):

A is a pyrrolidine ring;

R¹ is -L¹-X, wherein L¹ is C₂-C₄ alkylene and X is a point of attachment to the oligonucleotide;

R² and R³, together with the atoms to which they are attached, form a five-membered heterocyclyl having one nitrogen atom;

R⁴ is selected from C₁-C₆ alkyl and -L²-Z, wherein L² is C₂-C₄ alkylene and Z is —SO₃H;

q is 0; and

R⁶ is hydrogen.

In some embodiments, in the moiety of formula (Ia):

A is a morpholine ring;

R¹ is wherein L¹ is C₂-C₄ alkylene and X is a point of attachment to the oligonucleotide;

R² is C₁-C₄ alkyl and R³ is hydrogen; or R² and R³, together with the atoms to which they are attached, form a six-membered ring having one nitrogen atom and one oxygen atom;

R⁴ is selected from C₁-C₆ alkyl and -L²-Z, wherein L² is C₂-C₄ alkylene and Z is —SO₃H;

q is 0; and

R⁶ is hydrogen.

In some embodiments, the moiety of formula (Ia) is selected from:

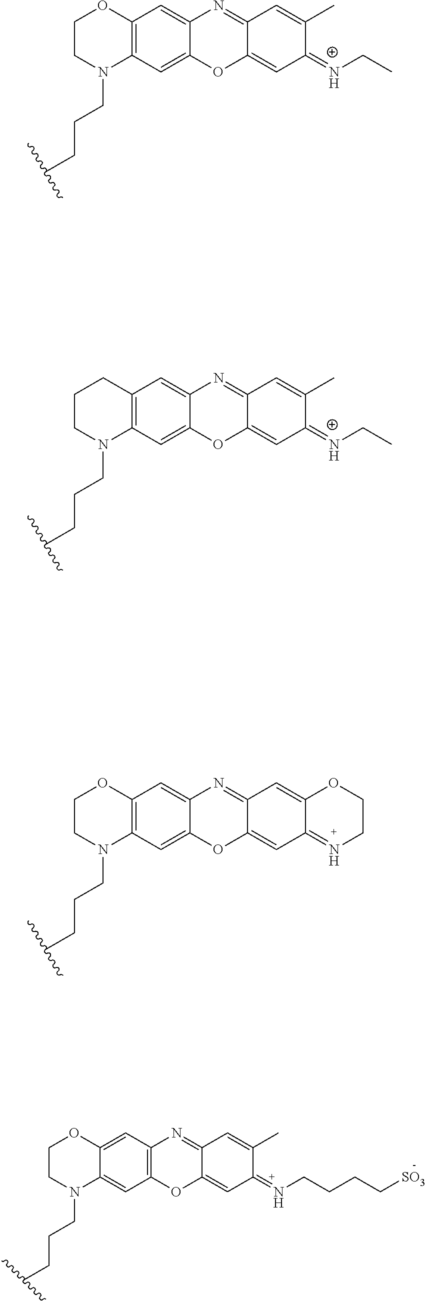

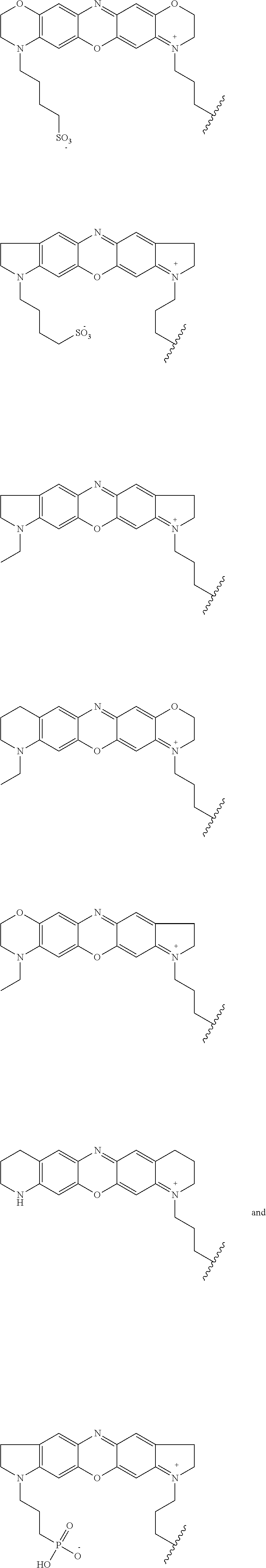

or a tautomer or a salt thereof,
wherein [symbol] represents the point of attachment of the moiety to the oligonucleotide.

Additional moieties of formula (Ia) include:

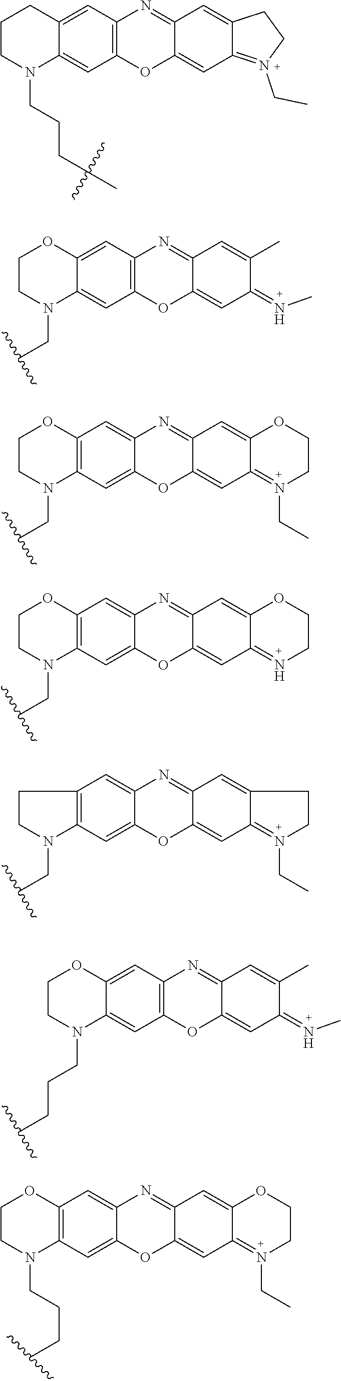

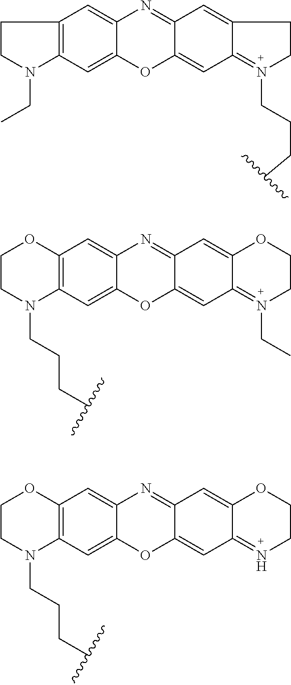

The moiety of formula (Ia) can be attached to the oligonucleotide via a direct bond, or via a linker. The linker can include one or more groups independently selected from methylene (—CH₂—), ethylene (—CH═CH—), ethynylene (—C≡C—), ether (—O—), amine (—NH—), thioether (—S—), carbonyl (—C(O)—), or sulfonyl (—S(O)₂—) moieties, or any combination thereof, such as an amide (—C(O)NH—), ester (—C(O)O—), carbamate (—OC(O)NH—), or sulfonamide (—S(O)₂NH—) group, and any combination thereof. The linker can also include one or more cyclic groups, such as an arylene, heteroarylene, cycloalkylene, or heterocycloalkylene moiety. For example, if a compound of formula (I) includes an alkyne or azide group and it is attached to an oligonucleotide via click chemistry, a triazole linking group will be formed and the linker will include a 1,2,3-triazole moiety. One skilled in the art will appreciate that copper-free click chemistry reactions can also be conducted, which would result in other types of linking moieties.

In some embodiments, the linker comprises one or more —(CH₂CH₂O)— (oxyethylene) groups, e.g., 1-20—(CH₂CH₂O)— groups (e.g., 1, 2, 3, 4, 5, 6, 7, 8, 9, 10, 11, 12, 13, 14, 15, 16, 17, 18, 19, or 20—(CH₂CH₂O)— groups, or any range therebetween). In some embodiments, the linker comprises a —(CH₂CH₂O)—, —(CH₂CH₂O)₂—, —(CH₂CH₂O)₃—, —(CH₂CH₂O)₄—, —(CH₂CH₂O)₅—, or —(CH₂CH₂O)₆— group.

In some embodiments, the linker comprises one or more alkylene groups (e.g., —(CH₂)ₙ—), wherein n is 1-12, e.g., 1, 2, 3, 4, 5, 6, 7, 8, 9, 10, 11, or 12, or any suitable range therebetween). In some embodiments, the linker comprises one or more branched alkylene groups.

In some embodiments, the linker comprises at least one amide group (—C(O)NH—). In some embodiments, the linker comprises two amide groups.

In some embodiments, the moiety of formula (Ia) is attached to the oligonucleotide via an amide moiety (—C(O)NH—). Such a linker may result following the reaction of a compound of formula (I) that comprises a succinimidyl ester group with an amine-modified oligonucleotide.

The labeled oligonucleotides can be synthesized according to standard methods. For example, a modified oligonucleotide containing a primary amino group can be attached to compounds of formula (I) that have a reactive moiety that reacts with primary amines, such as an active ester (e.g., a succinimidyl ester). Accordingly, in one aspect, disclosed herein is a method of synthesizing a labeled oligonucleotide, comprising reacting an oligonucleotide with a compound of formula (I) disclosed herein, to provide a labeled oligonucleotide (e.g., an oligonucleotide comprising a moiety of formula (Ia)).

The oligonucleotide can be of any suitable length, for example, a length suitable for use as a primer in a sequencing reaction. In some embodiments, the oligonucleotide is about 5 bases to about 50 bases in length, or any range therebetween, such as about 15 bases to about 35 bases in length. In some embodiments, the oligonucleotide is about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases in length, or any range therebetween.

The labeled oligonucleotides of the present disclosure can be used in sequencing methods, such as those described hereinbelow. For use in such methods, the disclosure also provides compositions comprising the labeled oligonucleotides. The compositions can further include one or more nucleic acid amplification reagents. In some embodiments, the one or more amplification reagents are selected from the group consisting of: deoxynucleotide triphosphates (e.g., unlabeled deoxynucleotide triphosphates), buffer, a magnesium salt (e.g., MgCl₂ or MgSO₄), a nucleic acid template, and a DNA polymerase (e.g., a thermostable DNA polymerase, such as Taq, Tca, Tfu, Tbr, Tth, Tih, Tfi, Tli, Tfl, Pfu, Pwo, KOD, Tma, Tne, Bst, Pho, Sac, Sso, or ES4, or a mutant, variant, or derivative of any thereof).

### Modified Nucleotide Triphosphate Compounds

Also disclosed herein are modified nucleotide triphosphate compounds that are labeled with the dye compounds disclosed herein, such as the dye compounds of formula (I). The term “modified” when used in connection with a “nucleotide triphosphate compound” indicates that the nucleotide triphosphate compound is covalently attached to a dye compound (such as a moiety of formula (Ib) described below), e.g., via a direct bond or via a linker, as discussed below.

For example, disclosed herein are modified nucleotide triphosphate compounds comprising a moiety of formula (Ib):

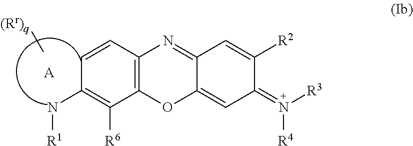

or tautomer or a salt thereof, wherein:

A is a five-, six-, or seven-membered heterocyclyl;

R¹ is selected from hydrogen, C₁-C₆ alkyl, C₁-C₆ heteroalkyl, and -L¹-X, wherein L¹ is alkylene or heteroalkylene, and X is selected from —COOH, —SO₃H, —PO₃H₂, —OPO₃H₂, and a point of attachment to the nucleotide triphosphate compound; or R¹ is taken together with R⁶ and the atoms to which they are attached to form a five-, six-, or seven-membered here;

R², R³, and R⁴ are defined follows:

(i) R² is C₁-C₄ alkyl; R³ is hydrogen; and R⁴ is selected from hydrogen, C₁-C₆ alkyl, C₁—C₆ heteroalkyl, and -L²-Z, wherein L² is alkylene or heteroalkylene, and Z is selected from —COOH, —SO₃H, —PO₃H₂, —OPO₃H₂, and a point of attachment to the nucleotide triphosphate compound; or

(ii) R² and R³, together with the atoms to which they are attached, form a five-, six-, or seven-membered heterocyclyl; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a point of attachment to the nucleotide triphosphate compound; or

(iii) R² is C₁-C₄ alkyl; and R³ and R⁴, together with the nitrogen atom to which they are attached, form a four-, five-, six-, or seven-membered heterocyclyl;

q is 0, 1, 2, or 3; and

each R⁵ is independently selected from C₁-C₄ alkyl; and

R⁶ is hydrogen, or R⁶ is taken together with R¹ and the atoms to which they are attached to form a five-, six-, or seven-membered heterocyclyl;

wherein either:
(c) R¹ is -L¹-X, and X is a point of attachment to the nucleotide triphosphate compound; or
(d) R⁴ is -L²-Z, and Z is a point of attachment to the nucleotide triphosphate compound.

In some embodiments, A is selected from a pyrrolidine, piperidine, and morpholine ring. In some embodiments, A is a morpholine ring. In some embodiments, A is a piperidine ring. In some embodiments, A is a pyrrolidine ring.

In some embodiments, R¹ is hydrogen, C₁-C₆ alkyl, or C₁-C₆ heteroalkyl. In some embodiments, R¹ is hydrogen or C₁-C₄ alkyl. In some embodiments, R¹ is hydrogen or ethyl. In some embodiments, R¹ is hydrogen. In some embodiments, R¹ is ethyl.

In some embodiments, R¹ is -L¹-X. In some embodiments, L¹ is C₂-C₄ alkylene or L¹ is C₂-C₄ heteroalkylene. In some embodiments, L¹ is C₂-C₄ alkylene. In some embodiments, L¹ is —CH₂CH₂CH₂—. In some embodiments, X is selected from —COOH, —SO₃H, —PO₃H₂, and a point of attachment to the nucleotide triphosphate compound. In some embodiments, X is selected from —SO₃H, —PO₃H₂, and a point of attachment to the nucleotide triphosphate compound. In some embodiments, X is —SO₃H. In some embodiments, X is —PO₃H₂. In some embodiments, X is a point of attachment to the nucleotide triphosphate compound.

In some embodiments, R² is C₁-C₄ alkyl, R³ is hydrogen, and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a point of attachment to the nucleotide triphosphate compound. In some embodiments, R² is C₁-C₄ alkyl, R³ is hydrogen, and R⁴ is selected from C₁-C₆ alkyl and -L²-Z, wherein L² is alkylene or heteroalkylene, and Z is —SO₃H. In some embodiments, R² is C₁-C₂ alkyl, R³ is hydrogen, and R⁴ is C₁-C₂ alkyl or —(CH₂)ₙ—SO₃H, wherein n is 3, 4, or 5. In some embodiments, R² is methyl, R³ is hydrogen, and R⁴ is methyl, ethyl or —(CH₂)₄—SO₃H. In some embodiments, R² is methyl, R³ is hydrogen, and R⁴ is ethyl. In some embodiments, R² is methyl, R³ is hydrogen, and R⁴ is —(CH₂)₄—SO₃H.

In some embodiments, R² and R³, together with the atoms to which they are attached, form a five-, six-, or seven-membered heterocyclyl; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a point of attachment to the nucleotide triphosphate compound. In some embodiments, R² and R³, together with the atoms to which they are attached, form a five-, six-, or seven-membered heterocyclyl; and R⁴ is selected from from hydrogen, C₁-C₆ alkyl, and C₁-C₆ heteroalkyl. In some embodiments, R² and R³, together with the atoms to which they are attached, form a five-, six-, or seven-membered heterocyclyl; and R⁴ is selected from hydrogen, and C₁-C₂ alkyl. In some embodiments, R² and R³, together with the atoms to which they are attached, form a five-, six-, or seven-membered heterocyclyl; and R⁴ is -L²-Z. In some embodiments, L² is C₂-C₄ alkylene. In some embodiments, L² is —CH₂CH₂CH₂—. In some embodiments, Z is selected from —COOH, —SO₃H, and a point of attachment to the nucleotide triphosphate compound.

In some embodiments, R² and R³, together with the atoms to which they are attached, form a five- or six-membered heterocyclyl; and R⁴ is selected from hydrogen, C₁-C₆ alkyl, C₁-C₆ heteroalkyl, and and -L²-Z, wherein L² is alkylene or heteroalkylene, and Z is selected from —COOH, —SO₃H, —PO₃H₂, —OPO₃H₂, and a point of attachment to the nucleotide triphosphate compound. In some embodiments, R² and R³, together with the atoms to which they are attached, form a five- or six-membered heterocyclyl; and R⁴ is selected from hydrogen and and -L²-Z, wherein L² is C₂-C₄ alkylene or C₂-C₄ heteroalkylene, and Z is a point of attachment to the nucleotide triphosphate compound. In some embodiments, R² and R³, together with the atoms to which they are attached, form a five- or six-membered heterocyclyl; and R⁴ is selected from hydrogen and -L²-Z, wherein L² is —CH₂CH₂CH₂—, and Z is a point of attachment to the nucleotide triphosphate compound.

In some embodiments, R² is C₁-C₄ alkyl; and R³ and R⁴, together with the nitrogen atom to which they are attached, form a four-, five-, six-, or seven-membered heterocyclyl. In some embodiments, R² is methyl; and R³ and R⁴, together with the nitrogen atom to which they are attached, form a four-, five-, six-, or seven-membered heterocyclyl.

In some embodiments, q is 0. In some embodiments, q is 1 and R⁵ is methyl.

In some embodiments, R⁶ is hydrogen.

In some embodiments, in the moiety of formula (Ib):

A is a pyrrolidine ring;

R¹ is -L¹-X, wherein L¹ is C₂-C₄ alkylene and X is a point of attachment to the nucleotide triphosphate compound;

R² and R³, together with the atoms to which they are attached, form a five-membered heterocyclyl having one nitrogen atom;

R⁴ is selected from C₁-C₆ alkyl and -L²-Z, wherein L² is C₂-C₄ alkylene and Z is —SO₃H;

q is 0; and

R⁶ is hydrogen.

In some embodiments, in the moiety of formula (Ib):

A is a morpholine ring;

R¹ is -L¹-X, wherein L¹ is C₂-C₄ alkylene and X is a point of attachment to the nucleotide triphosphate compound;

R² is C₁-C₄ alkyl and R³ is hydrogen; or R² and R³, together with the atoms to which they are attached, form a six-membered ring having one nitrogen atom and one oxygen atom;

R⁴ is selected from C₁-C₆ alkyl and -L²-Z, wherein L² is C₂-C₄ alkylene and Z is —SO₃H;

q is 0; and

R⁶ is hydrogen.

In some embodiments, the moiety of formula (Ib) is selected from:

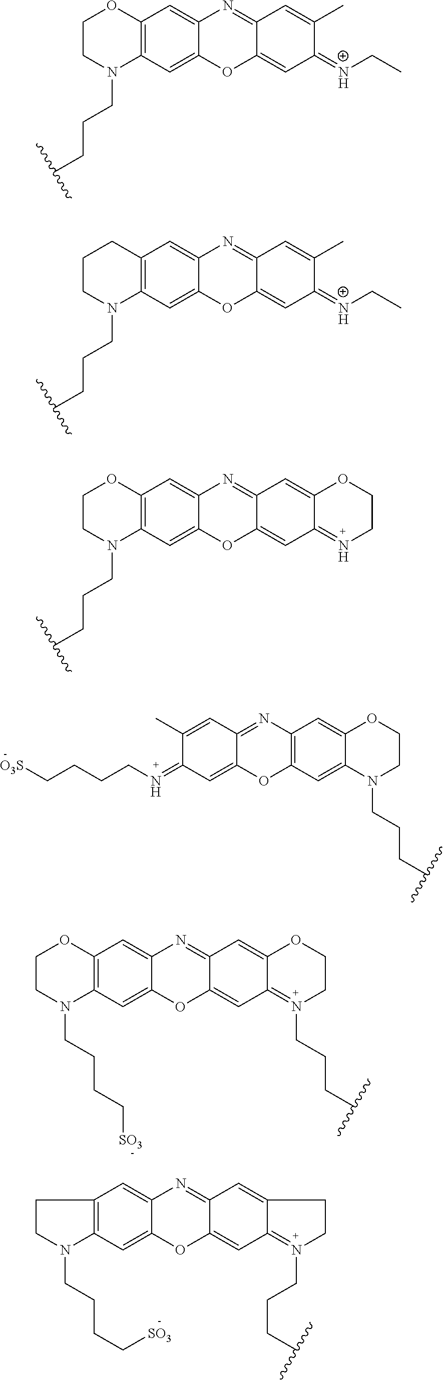

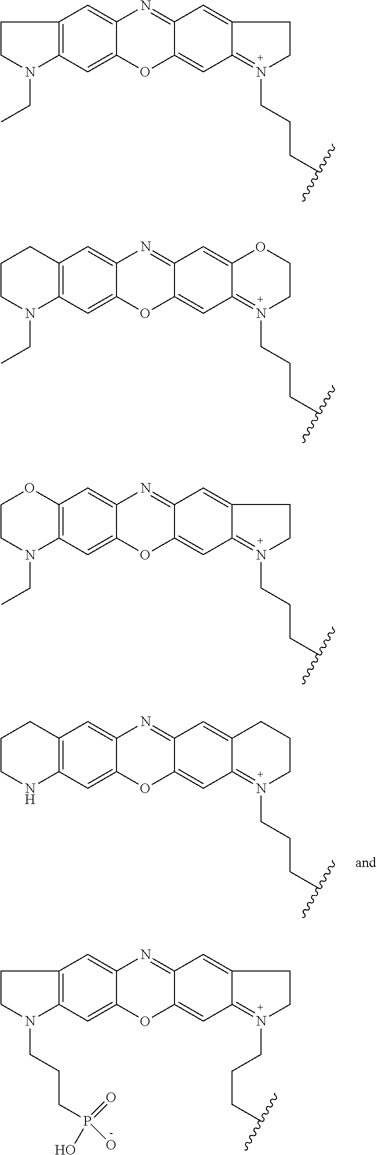

or a tautomer or a salt thereof,

wherein [symbol] represents the point of attachment of the moiety to the nucleotide triphosphate compound.

The moiety of formula (Ib) can be attached to the nucleotide triphosphate compound via a direct bond, or via a linker. The linker can include one or more groups independently selected from methylene (—CH₂—), ethylene (—CH═CH—), ethynylene (—C≡C—), ether (—O—), amine (—NH—), thioether (—S—), carbonyl (—C(O)—), or sulfonyl (—S(O)₂—) moieties, or any combination thereof, such as an amide (—C(O)NH—), ester (—C(O)O—), carbamate (—OC(O)NH—), or sulfonamide (—S(O)₂NH—) group, and any combination thereof. The linker can also include one or more cyclic groups, such as an arylene, heteroarylene, cycloalkylene, or heterocycloalkylene moiety. For example, if a compound of formula (I) includes an alkyne or azide group and it is attached to a nucleotide triphosphate compound via click chemistry, a triazole linking group will be formed and the linker will include a 1,2,3-triazole moiety. One skilled in the art will appreciate that copper-free click chemistry reactions can also be conducted, which would result in other types of linking moieties.

In some embodiments, the linker comprises one or more —(CH₂CH₂O)— (oxyethylene) groups, e.g., 1-20 —(CH₂CH₂O)— groups (e.g., 1, 2, 3, 4, 5, 6, 7, 8, 9, 10, 11, 12, 13, 14, 15, 16, 17, 18, 19, or 20 —(CH₂CH₂O)— groups, or any range therebetween). In some embodiments, the linker comprises a —(CH₂CH₂O)—, —(CH₂CH₂O)₂—, —(CH₂CH₂O)₃—, —(CH₂CH₂O)₄—, —(CH₂CH₂O)₅—, or —(CH₂CH₂O)₆— group.

In some embodiments, the linker comprises one or more alkylene groups (e.g., —(CH₂)ₙ—), wherein n is 1-12, e.g., 1, 2, 3, 4, 5, 6, 7, 8, 9, 10, 11, or 12, or any suitable range therebetween). In some embodiments, the linker comprises one or more branched alkylene groups.

In some embodiments, the linker comprises at least one amide group (—C(O)NH—). In some embodiments, the linker comprises two amide groups.

In some embodiments, the moiety of formula (Ia) is attached to the nucleotide triphosphate compound via an amide moiety (—C(O)NH—). Such a linker may result following the reaction of a compound of formula (I) that comprises a succinimidyl ester group with an amine-modified nucleotide triphosphate compound.

The modified nucleotide triphosphate compound can be a modified deoxynucleotide triphosphate compound or a modified dideoxynucleotide triphosphate compound. For example, in some embodiments, the compound is a modified deoxynucleotide triphosphate compound selected from deoxyadenosine triphosphate, deoxycytidine triphosphate, deoxyguanosine triphosphate, and deoxythymidine triphosphate. In some embodiments, the compound is a modified dideoxynucleotide triphosphate compound selected from dideoxyadenosine triphosphate, dideoxycytidine triphosphate, dideoxyguanosine triphosphate, and dideoxythymidine triphosphate. For example, exemplary modified deoxynucleotides include the following:

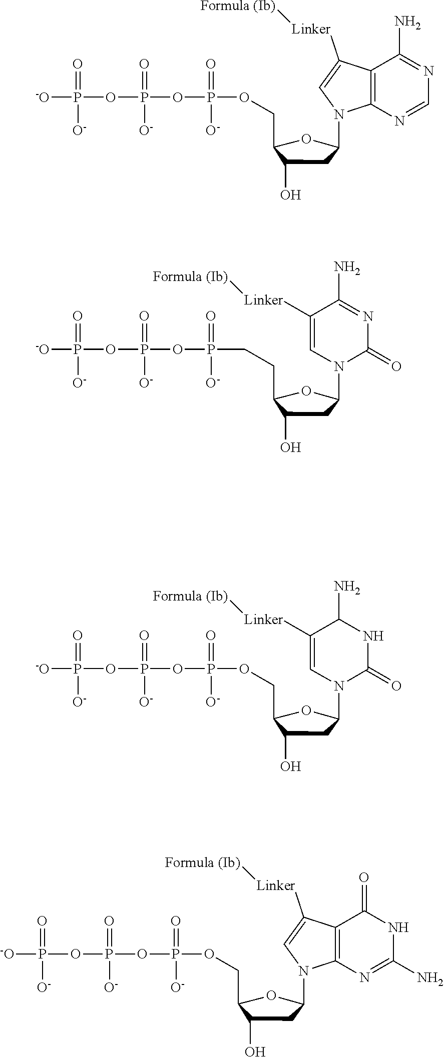

Exemplary modified dideoxynucleotides include the following:

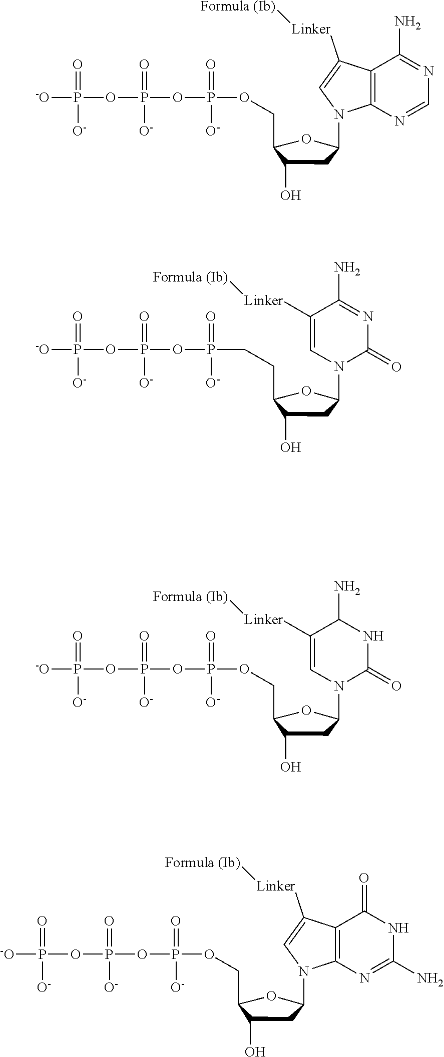

The modified nucleotide triphosphate compounds can be synthesized according to standard methods. For example, a modified nucleotide triphosphate compound containing a primary amino group can be attached to compounds of formula (I) that have a reactive moiety that reacts with primary amines, such as an active ester (e.g., a succinimidyl ester). Such Accordingly, in one aspect, disclosed herein is a method of synthesizing a labeled oligonucleotide, comprising reacting a nucleotide triphosphate compound (e.g., a nucleotide triphosphate compound functionalized with a linker) with a compound of formula (I) disclosed herein, to provide a labeled oligonucleotide (e.g., an oligonucleotide comprising a moiety of formula (Ia)).

The modified nucleotide triphosphate compounds of the present disclosure can be used in sequencing methods, such as those described hereinbelow. The modified nucleotide triphosphate compounds of the present disclosure can be used in sequencing methods, such as those described hereinbelow. For use in such methods, the disclosure also provides compositions comprising the modified nucleotide triphosphate compounds. The compositions can further include one or more nucleic acid amplification reagents. In some embodiments, the one or more amplification reagents are selected from the group consisting of: deoxynucleotide triphosphates (e.g., unlabeled deoxynucleotide triphosphates), buffer, a magnesium salt (e.g., MgCl₂ or MgSO₄), an oligonucleotide primer, a nucleic acid template, and a DNA polymerase (e.g., a thermostable DNA polymerase, such as Taq, Tca, Tfu, Tbr, Tth, Tih, Tfi, Tli, Tfl, Pfu, Pwo, KOD, Tma, Tne, Bst, Pho, Sac, Sso, or ES4, or a mutant, variant, or derivative of any thereof).

### Methods of Use

The compounds of the present disclosure can be used for any suitable molecular biology or biochemical assay that involves the detection and/or quantification of labeled nucleotides, oligonucleotides, and/or polynucleotides. As described further herein, the dyes of the present disclosure exhibit many advantageous features, including but not limited to, stability at elevated temperatures and longer wavelength emission. In some embodiments, the dyes of the present disclosure can be used to label individual nucleic acids (e.g., dNTPs) and/or oligonucleotides (e.g., primers/probes), which are useful for any methods involving nucleic acid amplification. For example, methods of nucleic acid amplification can include, but are not limited to, polymerase chain reaction (PCR), quantitative PCR, real time PCR, hot start PCR, single cell PCR, nested PCR, in situ colony PCR, digital PCR (dPCR), Droplet Digital™ PCR (ddPCR), emulsion PCR, ligase chain reaction (LCR), transcription based amplification system (TAS), nucleic acid sequence-based amplification (NASBA), strand displacement amplification (SDA), rolling circle amplification (RCA), and hyper-branched RCA (HRCA).

In accordance with these embodiments, RT-PCR generally refers to PCR in which the reverse transcription reaction that converts the target RNA into complementary single-stranded DNA is performed first, and then the DNA is amplified. Real-time PCR generally refers to PCR in which the amount of reaction product (target amplification product) is monitored as the reaction progresses. There are many forms of real-time PCR that differ primarily in the detection chemistry used to monitor reaction products, such as the dyes described further herein. Nested PCR generally refers to two-step PCR, where the amplification product of the first PCR becomes a sample for the second PCR with a new primer set, and at least one of these primers is inside the first amplification product. Multiplex PCR generally refers to PCR in which multiple target sequences (or a single target sequence and one or more reference sequences) are simultaneously performed in the same reaction mixture. Typically, different primer sets are used for each of the amplified sequences, including primer sets labeled with the dyes of the present disclosure. Quantitative PCR generally refers to PCR designed to measure the abundance of one or more specific target sequences in a sample or sample, which can include the use of the dyes of the present disclosure. Digital PCR generally refers to compartmentalizing a bulk PCR reaction into thousands of nanoliter-scale reactions, each containing zero, one, or just a few DNA molecules. By counting positive reactions based on probe fluorescence, including fluorescent signals produced by the dyes of the present disclosure, absolute quantification of the sample can be obtained. dPCR overcomes common limitations of qPCR, such as the need for standard curves, low accuracy when measuring rare targets, and lack of sensitivity in high background conditions.

In some embodiments, as described above, the dyes of the present disclosure can be used to label individual nucleic acids (e.g., dNTPs) and/or oligonucleotides (e.g., primers/probes) to detect, track, and/or quantify one or more target nucleic acids during or after amplification. In some embodiments, target nucleic amplification can be tracked, detected, and/or quantified directly via the labeled nucleic acids and/or oligonucleotides (e.g., qPCR, RT-PCR), and in other embodiments, target nucleic amplification can be tracked, detected, and/or quantified indirectly via the labeled nucleic acids and/or oligonucleotides (e.g., Taqman-based assays, strand displacement assays). As would be recognized by one of ordinary skill in the art based on the present disclosure, the dyes described herein can be used in any nucleic acid amplification and/or detection assay. In some embodiments, the dyes of the present disclosure can be used to label a nucleic acid, oligonucleotide sequences, single-stranded DNA, double-stranded DNA, RNA (e.g., mRNA or miRNA), or DNA-RNA hybrids. In some embodiments, the nucleic acid labeled using the dyes of the present disclosure is about 10, 15, 20, 25, 30, 35, 40, 45, 50, 60, 70, 80, 90, 100, 150, 200, 250, 300, 350, 400, 450, 500, 600, 700, 800, 900, or 1000 nucleotides in length. In some embodiments, the dyes of the present disclosure are used to label a sequence that is complementary or substantially complementary to a target sequence or another probe sequence. In some embodiments, the dyes of the present disclosure can be used with a quencher that anneals to the same target nucleic acid, or one that is complementary thereto, thereby enabling detection of the nucleic acid label.

Embodiments of the present disclosure include the use of the dyes described herein for nucleic acid sequencing applications, including but not limited to, fragment analysis and next generation sequencing. For example, in some embodiments, fragment analysis comprises a series of techniques in which DNA fragments are fluorescently labeled using the dyes of the present disclosure, separated by capillary electrophoresis (CE), and sized by comparison to an internal standard. While DNA sequencing by CE is used to determine the specific base sequence of a particular fragment or gene segment, fragment analysis can provide sizing, relative quantitation, and genotyping information for fluorescently labeled DNA fragments produced by PCR using primers designed for a specific DNA target.

In some embodiments, the dyes of the present disclosure can be used as part of methods to determine the series of base pairs in a DNA and/or RNA molecule (i.e., nucleic acid sequencing), including for use in whole-genome sequencing and region sequencing, transcriptome analysis, metagenomics, small RNA discovery, methylation profiling, and genome-wide protein-nucleic acid interaction analysis. For example, in some embodiments, replication of a DNA template strand proceeds with a reaction mixture including the four standard dNTPs and all four ddNTPs, each labelled with a different fluorescent dye (ddATP, ddCTP, ddGTP, and ddATP), such as those described in the present disclosure. Random incorporation of the labelled ddNTPs produces a series of DNA fragments in which chain growth has been terminated at each successive position, each one nucleotide longer than the previous. Separation of the fragments by size produces a sequencing ladder as a series of colored bands. In an automated DNA sequencer, the fluorescent dye of each band is activated by a scanning laser as it passes a set point at the bottom of the electrophoretic gel. The color of each successive band is read by a fluorometer, and a computer assembles these as a gel image, which can be read from bottom to top, like a conventional radioactively-labelled sequencing ladder. Multiple sequencing reactions on separate templates are run in parallel (the bands in each ladder are read as a separate electropherogram or chromatogram). The dyes of the present disclosure can be used with any currently available nucleic acid sequencing methods, including but not limited to, conventional Sanger sequencing or by “next generation sequencing” (NGS), which includes but is not limited to, sequencing-by-synthesis, sequencing-by-ligation, single molecule sequencing, nanopore-sequencing, and the like.

The following examples further illustrate aspects of the disclosure but, of course, should not be construed as in any way limiting its scope.

### EXAMPLES

The following abbreviations are used in the Examples: ACN is acetonitrile; AcOH is acetic acid; DCM is dichloromethane; DI is deionized; DIPEA is N,N-diisopropylethylamine; DMF is dimethylformamide; Et₂O is diethyl ether; EtOAc is ethyl acetate; HPLC is high performance liquid chromatography; LC-MS is liquid chromatography-mass spectrometry; LRMS is low resolution mass spectrometry; MeOH is methanol; NMR is nuclear magnetic resonance; RT is room temperature; TFA is trifluoroacetic acid; THF is tetrahydrofuran; TMSBr is bromotrimethylsilane; TSTU is N,N,N′,N′-tetramethyl-O—(N-succinimidyl)uronium tetrafluoroborate.

### Example 1: Compound Syntheses

### 5-(Ethylamino)-4-methyl-2-nitrosophenol

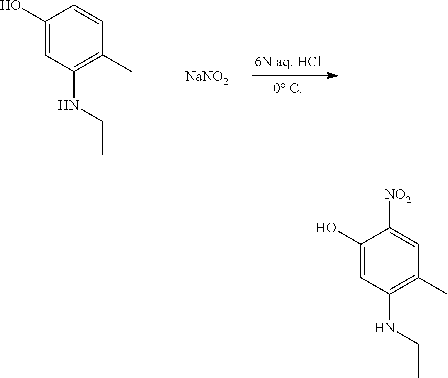

General Procedure 1: 3-Ethylamino-p-cresol (1.0 g, 6.6 mmol, 1.0 equiv) was dissolved in 5 mL ice-cold 6M HCl. To the solution, NaNO₂ (479 mg, 6.9, 1.05 equiv) was added in three portions over 1 h by keeping reaction in an ice bath. The reaction was stirred for another 2 h. Afterward, the precipitation was filtered through a funnel, washed with 15-20 mL 2M HCl, and dried via high vacuum to afford 5-(ethylamino)-4-methyl-2-nitrosophenol (1.1 g, 93%). LR-MS [M+H]⁺ 181.2.

### Ethyl 4-(6-methoxy-2,3-dihydro-4H-benzo[b] [1,4] oxazin-4-yl)butanoate

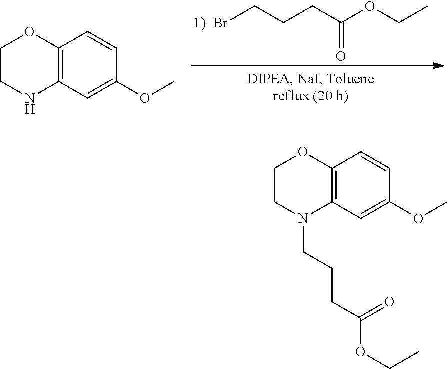

General Procedure 2: 6-Methoxy-3,4-dihydro-2H-benzo[b][1,4]oxazine (320 mg, 1.9 mmol, 1.0 equiv), Nal (29 mg, 0.19 mmol, 0.1 equiv), ethyl 4-bromobutanoate (809 μL 5.8 mmol, 3.0 equiv), and DIPEA (3.4 mL, 19.4 mmol, 10 equiv) were suspended in toluene (20 mL) and heated up to 120° C. for 20 h. The reaction was then cooled down and filtered over Celite under vacuum. Precipitation was washed with Et₂O/DCM (1/1, 50×2 mL). The filtrate was concentrated in vacuo, and the desired product was purified by silica gel purification. ¹H NMR (400 MHz, Chloroform-d) δ 6.68 (dd, J=9.1, 2.2 Hz, 1H), 6.29 (d, J=2.9 Hz, 1H), 6.15 (dd, J=8.7, 2.9 Hz, 1H), 4.15 (ddd, J=17.0, 6.3, 2.4 Hz, 4H), 3.75 (t, J=1.6 Hz, 3H), 3.40-3.18 (m, 4H), 2.36 (t, J=7.3 Hz, 2H), 1.94 (p, J=7.4 Hz, 2H), 1.25 (ddd, J=8.7, 7.0, 1.9 Hz, 3H). LRMS m/z: [M+H]⁺280.3.

### Ethyl 4-(7-hydroxy-3,4-dihydroquinolin-1(2H)-yl)butanoate

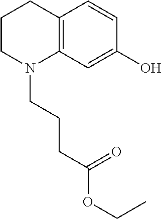 [SMILES: CCOC(=O)CCCN1CCCc2ccc(O)cc21]

The target compound was synthesized according to General Procedure 2, from 1,2,3,4-tetrahydroquinolin-7-ol. ¹H NMR (400 MHz, Chloroform-d) δ 6.80 (d, J=7.9 Hz, 1H), 6.22-5.96 (m, 2H), 4.56 (s, 1H), 4.26-4.04 (m, 2H), 3.27 (t, J=6.9 Hz, 4H), 2.69 (t, J=6.4 Hz, 2H), 2.38 (dd, J=8.1, 6.3 Hz, 2H), 1.94 (dd, J=10.0, 5.8 Hz, 4H), 1.29 (td, J=7.2, 1.7 Hz, 3H). LRMS m/z: [M+H]⁺264.3.

### Ethyl 4-(6-iodoindolin-1-yl)butanoate

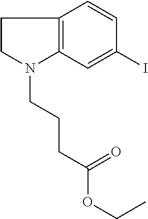 [SMILES: CCOC(=O)CCCN1CCc2ccc(I)cc21]

The target compound was synthesized according to General Procedure 2, from 6-iodoindoline. ¹H NMR (400 MHz, Chloroform-d) δ 6.95 (d, J=7.7 Hz, 1H), 6.78 (d, J=7.6 Hz, 1H), 6.73 (s, 1H), 4.15 (q, J=6.9 Hz, 3H), 3.38 (t, J=8.4 Hz, 2H), 3.09 (t, J=7.1 Hz, 2H), 2.92 (t, J=8.4 Hz, 2H), 2.42 (t, J=7.3 Hz, 2H), 1.93 (p, J=7.2 Hz, 2H), 1.28 (t, J=7.1 Hz, 4H). LRMS m/z: [M+H]⁺360.2.

### 4-(6-Methoxy-2,3-dihydro-4H-benzo[b] [1,4] oxazin-4-yl)butanoic acid

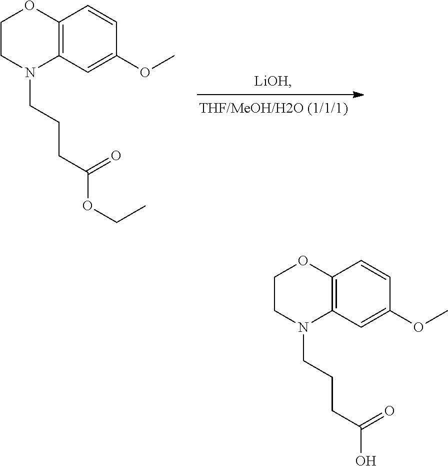 [SMILES: CCOC(=O)CCCN1CCOc2ccc(OC)cc21 ; "LiOH," ; COc1ccc2c(c1)N(CCCC(=O)O)CCO2]

General Procedure 3: Ethyl 4-(6-methoxy-2,3-dihydro-4H-benzo[b][1,4]oxazin-4-yl)butanoate (1.0 g, 3.6 mmol, 1.0 equiv) was dissolved in THF/MeOH (1/1.10 mL). 5 mL of 2 N LiOH was added, and the reaction mixture was stirred at RT for 3 h. LC-MS indicated full conversion. The volatile solvent was removed in vacuo, and the pH of the remaining solution was adjusted to pH 4.0 using 2 N aq. HCl. The suspension was then extracted with EtOAc (50×3 mL). Combined organic solution was concentrated in vacuo, and the desired product was purified by silica gel purification. ¹H NMR (400 MHz, Chloroform-d) δ 6.69 (dd, J=8.7, 2.1 Hz, 1H), 6.29 (t, J=2.6 Hz, 1H), 6.16 (dd, J=8.5, 3.1 Hz, 1H), 5.30 (t, J=1.7 Hz, 1H), 4.18 (dt, J=4.8, 2.7 Hz, 2H), 3.74 (d, J=1.7 Hz, 3H), 3.37-3.25 (m, 4H), 2.44 (td, J=7.0, 2.1 Hz, 2H), 1.95 (p, J=7.3 Hz, 2H). LRMS m/z: [M−H]⁻ 250.3.

### (Z)-4-(8-(Ethyliminio)-9-methyl-2,3-dihydro-[1,4]oxazino[2,3-b]phenoxazin-4(8H)-yl)butanoate

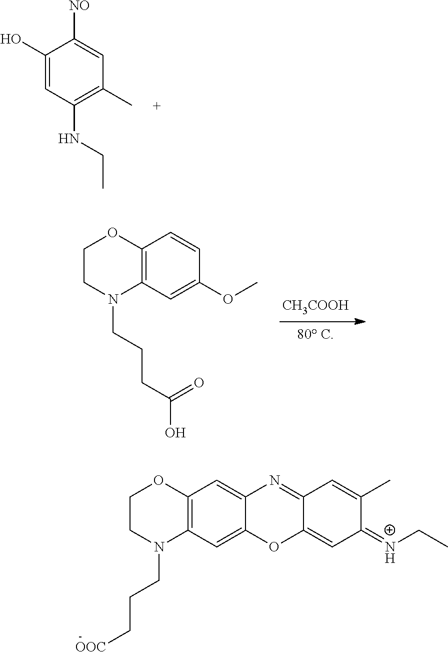

General Procedure 4: 4-(6-methoxy-2,3-dihydro-4H-benzo[b][1,4]oxazin-4-yl)butanoic acid (86 mg, 0.34 mmol, 1.0 equiv) and 5-(ethylamino)-4-methyl-2-nitrosophenol (61 mg, 0.34 mmol, 1.0 equiv) was dissolved in AcOH (4 mL). The suspension was heated up to 80° C. and stayed for 30 min. LC-MS indicated full consumption of the reactants. The reaction mixture turned deep blue and concentrated in vacuo. The desired product was purified by silica gel purification using DCM/MeOH/1% DIPEA. ¹H NMR (400 MHz, Methanol-d₄) δ 7.62 (s, 1H), 7.31 (s, 1H), 7.22 (s, 1H), 6.93 (s, 1H), 4.38 (d, J=5.0 Hz, 2H), 3.95-3.69 (m, 4H), 3.60 (d, J=7.3 Hz, 2H), 2.34 (d, J=21.6 Hz, 5H), 2.05 (t, J=8.1 Hz, 2H), 1.39 (t, J=7.3 Hz, 5H). LRMS m/z: [M+H]⁺382.4.

Compound JC-0025

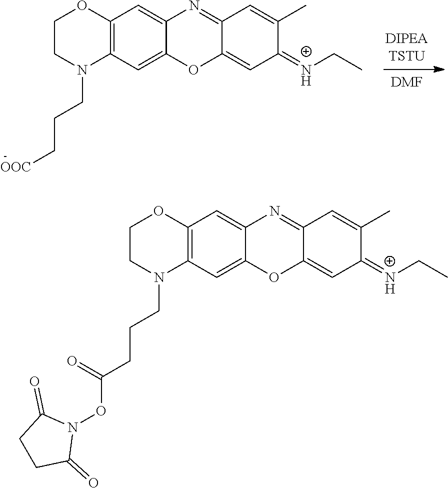

General Procedure 4: (Z)-4-(8-(ethyliminio)-9-methyl-2,3-dihydro-[1,4]oxazino[2,3-b]phenoxazin-4(8H)-yl)butanoate (38 mg, 1.0 mmol, 1.0 equiv) was mixed with TSTU (60 mg, 2.0 mmol, 2.0 equiv) and DIPEA (870 μL, 5.0 mmol, 5.0 equiv) in dry DMF (2 mL). The reaction was stirred at RT for 30 min. LC-MS indicated complete conversion. The desired product was purified using reverse preparative HPLC (ACN/0.5% TFA in DI H₂O). LRMS m/z: [M]⁺479.74.

Additional Compounds

Compounds JC-0028, JC-0064, JC-0081, JC-0084, CS-1333, CS-1345 and JC-0068 were synthesized analogously to compound JC-0025, according to General Procedure 4 and General Procedure 5, and using appropriate starting materials. Mass spec data are provided in Table 1 below.

### (E)-4-(6-Hydroxy-7-((4-nitrophenyl)diazenyl)-2,3-dihydro-4H-benzo[b][1,4]oxazin-4-yl)butanoic acid

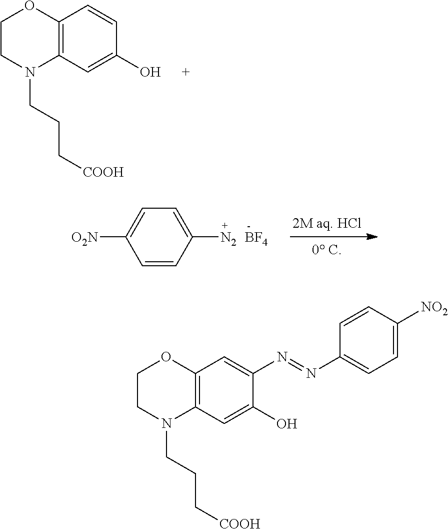

General Procedure 6: 4-(6-hydroxy-2,3-dihydro-4H-benzo[b][1,4]oxazin-4-yl)butanoic acid (145 mg, 0.61 mmol, 1 equiv) was dissolved in 2N HCl (2 mL) at 0° C. To the solution, 4-nitrobenzenediazonium tetrafluoroborate (173 mg, 0.73 mmol, 1.2 equiv) was added by keeping reaction in an ice bath. The reaction was stirred for another 1 h. Afterward, the precipitation was filtered through a funnel, washed with 2M HCl, and dried via high vacuum to afford product (206 mg, 87%). LRMS m/z: [M+H]⁺388.2.

### 4-(6-Hydroxy-2,3-dihydro-4H-benzo[b][1,4]oxazin-4-yl)butane-1-sulfonic acid

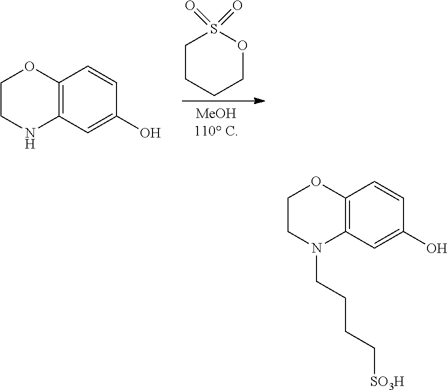 [SMILES: Oc1ccc2c(c1)NCCO2 ; O=S1(=O)CCCCO1 ; O=S(=O)(O)CCCCN1CCOc2ccc(O)cc21]

General Procedure 7: 3,4-dihydro-2H-benzo[b][1,4]oxazin-6-ol (200 mg, 1.3 mmol, 1.0 equiv) and 1,4-Butane sultone (360 mg, 2.7 mmol, 2.0 equiv) were added into a 25 mL pressure flask with a magnetic stir bar. The mixture was heated at 110° C. After about 30 min, the reaction mixture became very viscous, and the stirring was discontinued. At which timepoint, the flask was cooled to RT, and the yellow solid was carefully broken into large pieces using a spatula. MeOH (3 mL) was added into the flask and the reaction was proceeded at 110° C. for another 5 h. The mixture was cooled to RT. The white crystal product (195 mg. 51%) was filtered out, washed with MeOH, and dried in vacuo. LRMS [M+H]⁺ 288.54.

### 4-((5-Hydroxy-2-methylphenyl)amino)butane-1-sulfonic acid

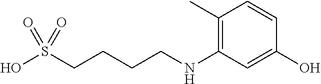 [SMILES: Cc1ccc(O)cc1NCCCCS(=O)(=O)O]

The desired product was synthesized analogously following General Procedure 7. 11-1 NMR (400 MHz, Chloroform-d) δ 6.94 (t, J=7.1 Hz, 2H), 6.34-6.04 (m, 4H), 3.65 (d, J=1.9 Hz, 3H), LRMS [M+H]⁺ 274.55.

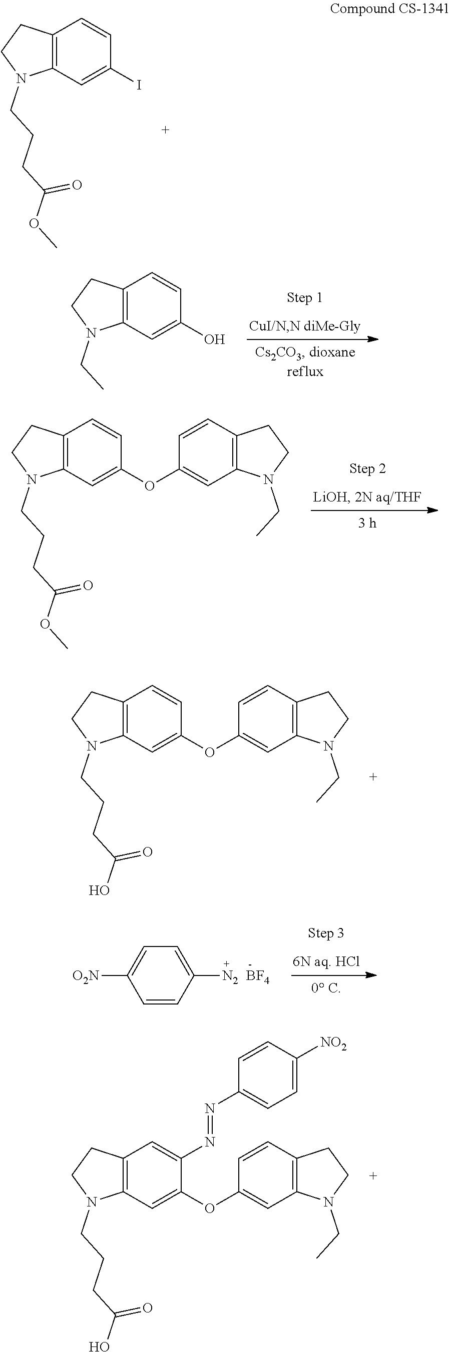

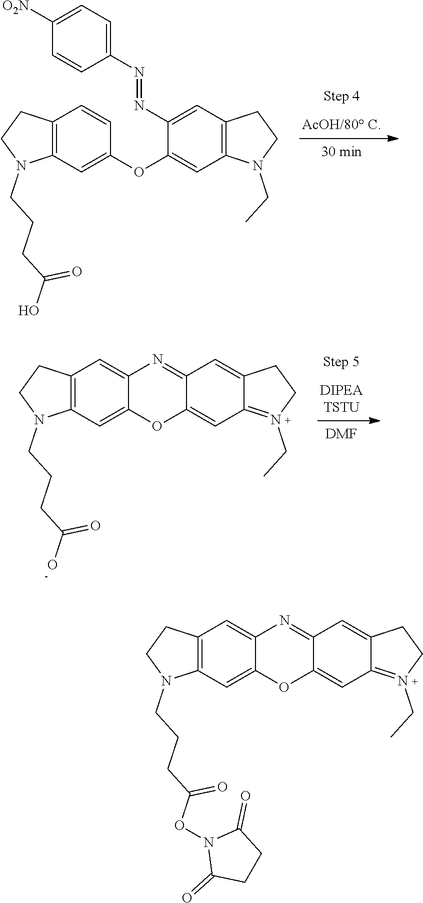

Step 1: A mixture of ethyl 4-(6-hydroxyindolin-1-yl)butanoate (580 mg, 2.5 mmol, 1.0 equiv), 1-ethyl-6-iodoindoline (785 mg, 3.2 mmol, 1.3 equiv), CuI (140 mg, 0.74 mmol, 0.3 equiv), N, N-dimethyl glycine (287 mg, 2.8 mmol, 1.1 equiv), and Cs₂CO₃ (2.4 g, 7.4 mmol, 3.0 equiv) was purged with Ar and suspended in dioxane (6 mL) in a sealed tube. The reaction was then heated at 100° C. for 20 h. The mixture was then cooled down and partitioned between EtOAc (100 mL) and DI H₂O (50 mL). The organic layer was separated, and the aqueous layer was extracted with EtOAc (3×50 mL). The combined organic layers were washed with brine, dried over Na₂SO₄, and concentrated in vacuo. The crude was purified by silica gel purification using Heptane/EtOAc as eluents to afford the desired product methyl 4-(6-((1-ethylindolin-6-yl)oxy)indolin-1-yl)butanoate. ¹H NMR (400 MHz, Chloroform-d) δ 6.94 (t, J=7.1 Hz, 2H), 6.34-6.04 (m, 4H), 3.65 (d, J=1.9 Hz, 3H), 3.48-3.26 (m, 4H), 3.20-2.99 (m, 4H), 2.92 (t, J=8.3 Hz, 4H), 2.39 (td, J=7.4, 2.0 Hz, 2H), 1.91 (p, J=7.0 Hz, 2H), 1.15 (t, J=7.4 Hz, 3H). LRMS m/z: [M+H]⁺380.2.

Step 2: To a solution of methyl 4-(6-((1-ethylindolin-6-yl)oxy)indolin-1-yl)butanoate (70 mg, 0.18 mmol, 1.0 equiv) in THF (4 mL), was added LiOH (22 mg, 0.46 mmol, 5 equiv) in 2 mL of DI H₂O. The solution was stirred at RT for 3 h. LC-MS indicated full conversion. The volatile solvent was then removed under vacuo and the aqueous was diluted with 20 mL H₂O. The pH of the aqueous solution was adjusted to pH 4-5. The suspension was then partitioned between EtOAc (100 mL) and DI H₂O. The aqueous layer was extracted with EtOAc (20×3 mL). The combined organic layers were then washed with H₂O (30 mL), brine (30 mL), dried over Na₂SO₄, and concentrated to afford the crude, 4-(6-((1-ethylindolin-6-yl)oxy)indolin-1-yl)butanoic acid, which was used in the next step without further purification.

Step 3: The desired mixtures of diazenyl intermediates were synthesized analogously following General Procedure 6. The crude was used in the next step without further purification.

Step 4: The diazenyl mixture was dissolved in AcOH (0.05 M) and heated at 80° C. for 30 min. The desired product, 4-(1-ethyl-2,3,7,8-tetrahydro-1H-dipyrrolo[3,2-b:2′,3′-i]phenoxazin-9-ium-9-yl)butanoate was purified by reverse phase HPLC using ACN/0.1% TFA in H₂O as mobile phases. ¹H NMR (400 MHz, Acetonitrile-d₃) δ7.42 (s, 2H), 6.61 (d, J=6.0 Hz, 2H), 4.01 (q, J=7.2 Hz, 4H), 3.61 (dt, J=16.6, 7.7 Hz, 4H), 3.27 (t, J=7.4 Hz, 4H), 2.45 (m, 4H), 1.31 (t, J=7.1 Hz, 3H). LRMS m/z: [M+H]⁺ 378.5.

Step 5: CS-1341 was synthesized from the product of the Step 4, following General Procedure 5. Mass spec data is provided below in Table 1.

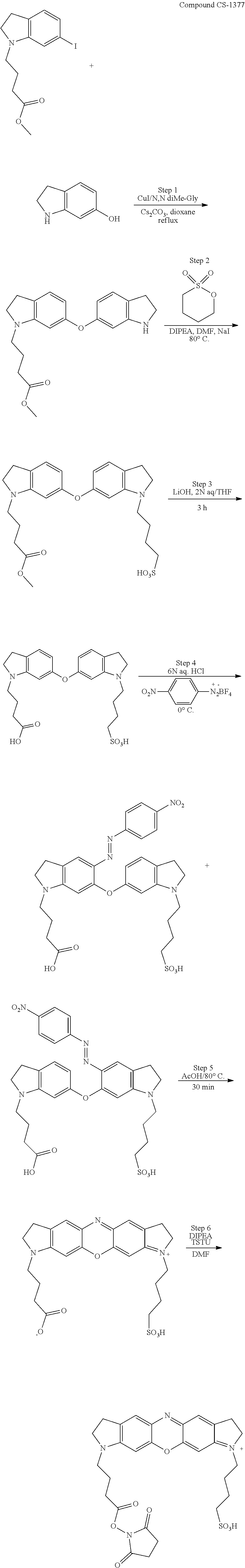

Step 1: A mixture of ethyl 4-(6-hydroxyindolin-1-yl)butanoate (84 mg, 0.36 mmol, 1.0 equiv), 6-iodoindoline (131 mg, 0.54 mmol, 1.5 equiv), CuI (20 mg, 0.11 mmol, 0.3 equiv), N, N-dimethyl glycine (33 mg, 0.33 mmol, 0.9 equiv), and Cs₂CO₃ (350 mg, 1.1 mmol, 3.0 equiv) was purged with Ar and suspended in dioxane (4 mL) in a sealed tube. The reaction was then heated at 100° C. for 20 h. The mixture was then cooled down and partitioned between EtOAc (100 mL) and DI H₂O (50 mL). The organic layer was separated, and the aqueous layer was extracted with EtOAc (3×50 mL). The combined organic layers were washed with brine, dried over Na₂SO₄, and concentrated in vacuo. The crude was purified by silica gel purification using Heptane/EtOAc as eluents to afford the desired product methyl 4-(6-(indolin-6-yloxy)indolin-1-yl)butanoate. 1H NMR (400 MHz, Methylene Chloride-d₂) δ6.99 (dd, J=19.4, 7.8 Hz, 2H), 6.29 (d, J=9.6 Hz, 2H), 6.21 (d, J=7.9 Hz, 1H), 6.15 (s, 1H), 3.65 (d, J=1.8 Hz, 3H), 3.58 (t, J=8.4 Hz, 2H), 3.41 (t, J=8.2 Hz, 2H), 3.16-2.82 (m, 6H), 2.41 (t, J=7.4 Hz, 2H), 1.92 (p, J=7.2 Hz, 2H). LRMS m/z: [M+H]⁺ 353.4.

Step 2: Target intermediate was synthesized following General Procedure 7. The desired product was isolated as DIPEA salt. ¹H NMR (400 MHz, Methanol-d₄) δ 6.93 (d, J=7.8 Hz, 2H), 6.24-6.06 (m, 4H), 3.75 (dd, J=7.1, 5.3 Hz, 1H), 3.61 (d, J=2.0 Hz, 3H), 3.43-3.35 (m, 4H), 3.28-3.18 (m, 1H), 3.03 (dt, J=10.1, 5.1 Hz, 4H), 2.87 (dt, J=16.3, 8.5 Hz, 7H), 2.41 (td, J=7.2, 2.0 Hz, 2H), 1.99-1.81 (m, 5H), 1.74 (q, J=7.5 Hz, 2H), 1.48-1.20 (m, 11H). LRMS m/z: [M−H]⁻ 487.2.

Step 3: Target intermediate was synthesized analogously following General Procedure 3. ¹H NMR (400 MHz, Deuterium Oxide) δ7.44 (d, J=7.9 Hz, 2H), 7.13 (dd, J=9.7, 4.1 Hz, 4H), 4.10-3.84 (m, 4H), 3.47 (q, J=6.8 Hz, 4H), 3.25 (q, J=7.6, 5.4 Hz, 4H), 2.90-2.69 (m, 2H), 2.42 (td, J=7.1, 2.2 Hz, 2H), 2.07-1.57 (m, 6H). LRMS m/z: [M−H]⁻ 473.2.

Step 4: The desired mixtures of diazenyl intermediates were synthesized analogously following General Procedure 6. The crude was used in the next step without further purification.

Step 5: The diazenyl mixture was dissolved in AcOH (0.05 M) and heated at 80° C. for 30 min. The desired product, 4-(9-(3-carboxypropyl)-2,3,7,8-tetrahydro-1H-dipyrrolo[3,2-b:2′,3′-i]phenoxazin-9-ium-1-yl)butane-1-sulfonate, was purified by silica gel purification using DCM/1% DIPEA in MeOH as eluents. ¹H NMR (400 MHz, Methanol-d₄) δ 7.32 (s, 2H), 6.67 (d, J=8.8 Hz, 2H), 3.98 (d, J=7.4 Hz, 4H), 3.70 (q, J=6.6 Hz, 1H), 3.59 (d, J=6.8 Hz, 3H), 3.28-3.12 (m, 4H), 2.86 (t, J=6.8 Hz, 2H), 2.38 (t, J=6.9 Hz, 2H), 2.13-1.70 (m, 7H), 1.36 (d, J=6.4 Hz, 6H). LRMS m/z: [M−H]⁻ 500.2.

Step 6: CS-1377 was synthesized analogously following General Procedure 5. Mass spec data is provided below in Table 1.

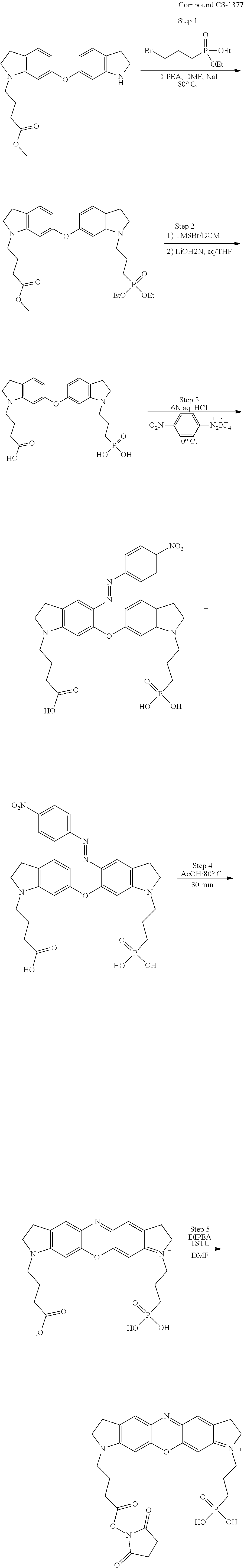

Step 1: Target intermediate was synthesized analogously following General Procedure 7 using diethyl (3-bromopropyl)phosphonate as the alkylating reagent. ¹H NMR (400 MHz, Chloroform-d) δ 6.96 (t, J=5.9 Hz, 2H), 6.33-6.09 (m, 4H), 4.12 (p, J=7.4 Hz, 5H), 3.67 (s, 3H), 3.46-3.32 (m, 4H), 3.07 (q, J=7.3, 6.9 Hz, 4H), 2.94 (t, J=8.4 Hz, 4H), 2.42 (t, J=7.4 Hz, 2H), 1.99-1.76 (m, 7H), 1.35 (q, J=6.9 Hz, 8H). LRMS m/z: [M+H]⁺ 531.6.

Step 2: To a solution of methyl 4-(6-((1-(3-(diethoxyphosphoryl)propyl)indolin-6-yl)-oxy)indolin-1-yl)butanoate (100 mg, 0.19 mmol, 1.0 equiv) in DCM (2 mL), TMSBr (2 mL) was added dropwise. The solution was stirred at RT for 20 h. LC-MS indicated full conversion to the corresponding phosphonic acid. The reaction was then concentrated in vacuo and used in the next step without further purification.

The crude product from the previous step was dissolved in THF/MeOH (1/1.4 mL). 2 mL of 2 N LiOH was added, and the reaction mixture was stirred at RT for 3 h. LC-MS indicated full conversion. The volatile solvent was removed in vacuo and pH of the remaining solution was adjusted to pH 4.0 using 2 N aq. HCl. The suspension was then extracted with EtOAc (50×3 mL). Combined organic solution was concentrated in vacuo, and the desired product was purified by silica gel purification. ¹H NMR (400 MHz, Methanol-d₄) δ 6.92 (d, J=7.8 Hz, 2H), 6.26-6.07 (m, 4H), 3.45-3.32 (m, 6H), 3.04 (q, J=6.3 Hz, 4H), 2.88 (t, J=8.3 Hz, 4H), 2.38 (t, J=7.3 Hz, 2H), 1.87 (p, J=7.6 Hz, 4H), 1.65 (dt, J=16.7, 7.9 Hz, 3H), 1.37 (d, J=6.5 Hz, 5H). LRMS m/z: [M+H]⁺ 481.5.

Step 3: The desired mixtures of diazenyl intermediates were synthesized analogously following General Procedure 6. The crude was used in the next step without further purification.

Step 4: The diazenyl mixture was dissolved in AcOH (0.05 M) and heated at 80° C. for 30 min. The desired product, 4-(1-(3-phosphonopropyl)-2,3,7,8-tetrahydro-1H-dipyrrolo[3,2-b:2′,3′-i]phenoxazin-9-ium-9-yl)butanoate, was purified by reverse HPLC using ACN/0.1% TFA in DI H₂O as mobile phases. 1H NMR (400 MHz, Methanol-d₄) δ 7.47 (s, 2H), 6.80 (d, J=14.8 Hz, 2H), 4.07 (d, J=9.1 Hz, 4H), 3.70 (dt, J=19.7, 7.4 Hz, 4H), 2.46 (t, J=6.8 Hz, 2H), 2.22-1.95 (m, 5H), 1.81 (dt, J=16.6, 7.8 Hz, 2H). LRMS m/z: [M+H]⁺ 472.5.

Step 5: CS-1480 was synthesized analogously following General Procedure 5. Mass spec data are provided in Table 1 below.

Characterization data, including mass spectrometry data and excitation and emission maxima, are shown in Table 1.

TABLE 1

| Compound# | Structure | Ex/nm | Em/nm | MS |
|---|---|---|---|---|
| JC-0025 | 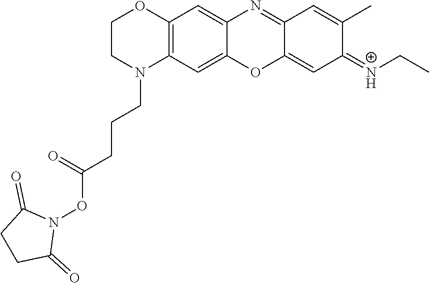 [SMILES: CC/[NH+]=c1/cc2oc3cc4c(cc3nc-2cc1C)OCCN4CCCC(=O)ON1C(=O)CCC1=O] | 635 | 658 | [M]⁺ 479.74 |
| JC-0028 | 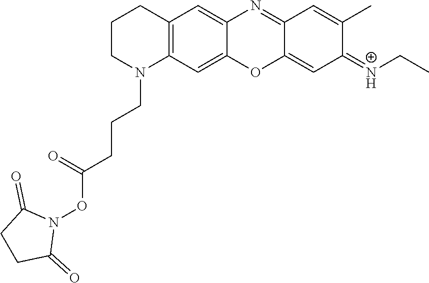 [SMILES: CC/[NH+]=c1/cc2oc3cc4c(cc3nc-2cc1C)CCCN4CCCC(=O)ON1C(=O)CCC1=O] | 632 | 640 | [M]⁺ 477.65 |
| JC-0064 | 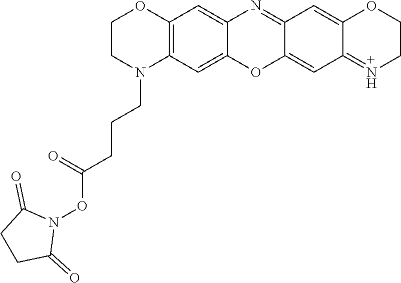 [SMILES: O=C(CCCN1CCOc2cc3c(cc21)Oc1cc2c(cc1=N3)OCCN=2)ON1C(=O)CCC1=O] | 640 | 657 | [M]⁺ 479.55 |
| JC-0081 | 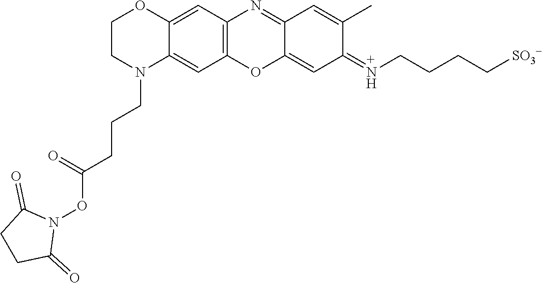 | 632 | 658 | [M + H]⁺ 587.53 |
| JC-0084 | 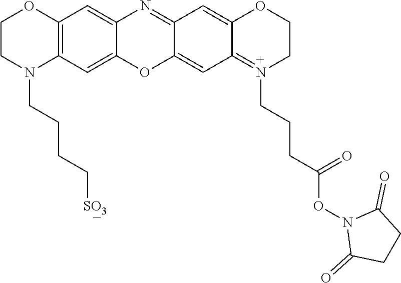 | 656 | 675 | [M + H]⁺ 615.47 |
| CS-1377 | 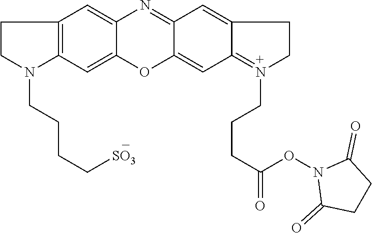 | 648 | 662 | [M + H]⁺ 583.19 |
| CS-1341 | 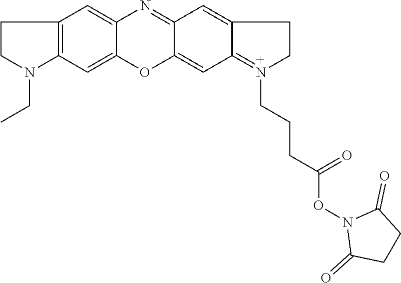 | 648 | 662 | [M + H]⁺ 615.47 |
| CS-1333 | 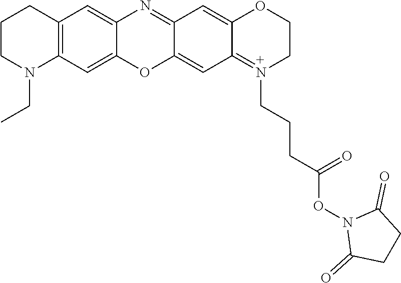 | 662 | 679 | [M + H]⁺ 615.47 |
| CS-1345 | 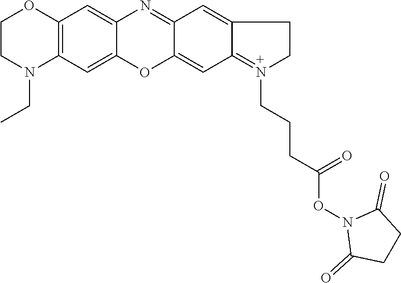 | 650 | 678 | [M]⁺ 475.20 |
| JC-0068 | 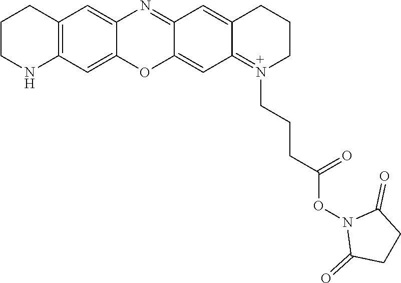 | 644 | 660 | [M]⁺ 475.55 |
| CS-1480 | 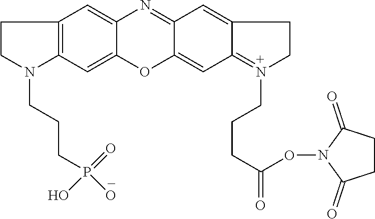 | 645 | 660 | [M + H]⁺ 569.18 |

### Example 2: General Procedures for Use of Oxaxine Dye N-Hydroxysuccinimidyl Esters Conjugating to Oligonucleotides

A. 1 μMole Scale. 5′-amino labeled or internal amino-deoxyuridine oligonucleotide was synthesized on an ABI 394 DNA synthesizer (1 μmole) using 5′Amino modifier C6 TFA amidite from Glen Research or Aminoallyl-dU amidite from PBI. Deprotection in concentrated ammonium hydroxide overnight at 60° C. yielded the amino-labeled oligonucleotide. The resulting oligonucleotide was evaporated to dryness, redissolved in 1 ml 2M NaCl (performed for counter-ion exchange), and desalted on NAP-10 size exclusion cartridge (GE Healthcare). After desalting, the oligonucleotide was evaporated to dryness followed by re-dissolution in 200 μl 0.5M sodium carbonate buffer, pH 8.5. The succinimidyl ester dye (JC-0025, JC-0081, CS-1341, CS-1377, or JC-0084) was dissolved in DMF at a concentration of 20 μl/mg. Two 20 μl aliquots of the dye/DMF solution were added to the dissolved oligonucleotide, 30 minutes apart. After the second addition, the reaction was mixed for 1 hour. After one hour, it was diluted to 1 ml with water and desalted on a NAP-10 column (GE Healthcare). The NAP-10 eluate was purified by reversed phase HPLC on a Phenomonex Jupiter C18 column using an acetonitrile/0.1M TEAA buffer system. The HPLC purified oligonucleotide was evaporated to dryness redissolved in 0.01M triethylammonium bicarbonate and desalted on a NAP-10 column. After final desalt step, the oligonucleotide was evaporated to dryness.

B. 100 μmole scale. The 5′-amino labeled or internal amino-deoxyuridine oligonucleotide was synthesized on an AKTA OligoPilot (100 μmole) DNA synthesizer using 5′ Amino modifier C6 TFA amidite from Glen Research or Aminoallyl dU amidite from PBI. Deprotection in concentrated ammonium hydroxide overnight at 60° C. yielded the 5′-aminohexyl labeled oligonucleotide. The resulting oligonucleotide was evaporated to dryness, redissolved in 75 ml 2M NaCl, and desalted on a 500 ml G-25 column (GE Healthcare). After desalting, the oligonucleotide was evaporated to dryness followed by re-dissolution in 50 ml 0.5M sodium carbonate buffer, pH 8.5. The succinimidyl ester dye (JC-0025, JC-0081, CS-1341, CS-1377, or JC-0084) was dissolved in DMF at a concentration of 20 μl/mg. 2400 μl of the dye/DMF solution was added dropwise to the dissolved oligonucleotide. The reaction was mixed for 1 hour. The dye conjugated oligonucleotide was neutralized with sodium acetate, pH 5.5 solution and precipitated from 2× volume of ethanol. The precipitated oligonucleotide was centrifuged at 9000 rpm for 60 minutes. The supernatant was decanted to waste. The resulting solid was dissolved in 70 ml water and purified by ion-exchange chromatography. The oligonucleotide was concentrated and desalted using tangential flow ultrafiltration and subsequently evaporated to dryness.

### Example 3: Multiplex PCR of STRs Using Dyes of the Present Disclosure

Experiments were conducted during development of embodiments of the present disclosure to determine the energy transfer characteristics of exemplary dyes JC-0025 and JC-0081 for potential use in an 8-dye multiplex PCR of STRs (short tandem repeats).

Oligonucleotides (oligos) were made as described above using the dyes JC-0025 and JC-0081 to derive primer pairs for D8S1179, FGA, and DYS385a/b loci. The oligos were made with either a 2 nucleotide spacer or 4 nucleotide spacer.

The primer pairs were then used in a triplex mix to amplify 2800M DNA. The oligos were then used in amplification reactions that were then analyzed on a Spectrum CE device.

The following amplification reaction conditions were used for each 25 ul reaction:
5× Master Mix: 5 ul
Nanopure water: 12.5 ul
5× Primer Pair (final concentrations: D8S1179: 1.0 uM; FGA: 3.20 uM; and DYS385a/b: 2.50 uM): 5.0 ul

The reaction mix was vortexed and dispensed into wells of a 96-well plate. 2.5 ul of 2800M DNA was then added to each well. Also included in the reactions: 4 replicates of 2800M and 2 replicate no template (water) controls. The reaction mixes were stored at 4° C. during thermal cycling then run on the CE as “No Amp”.

The reactions were run in an amplification reaction in a ProFlex Thermal cyclers follows:

1 cycle:
96° C. for 1 minute then
29 cycles:
98° C. for 5 seconds
60° C. for 1 min
72° C. for 15 seconds
final extension:
60° C. for 10 minutes
4° C. soak

Amplified samples were then analyzed with a Spectrum CE Beta 02 device. 10 ul of ILS (0.25 CCO ILS 2× Fragment Mix and 9.75 HiDi) were added to each well of a 96-well plate. 1 ul of the amplification reaction or No Amp mix was then added to the appropriate well. The plate was spun briefly to remove bubbles, denatured in a thermal cycler for 3 minutes, and then placed on ice for at least 3 minutes. Samples were injected using a 2 kV, 15 second injection with 8C spectral.

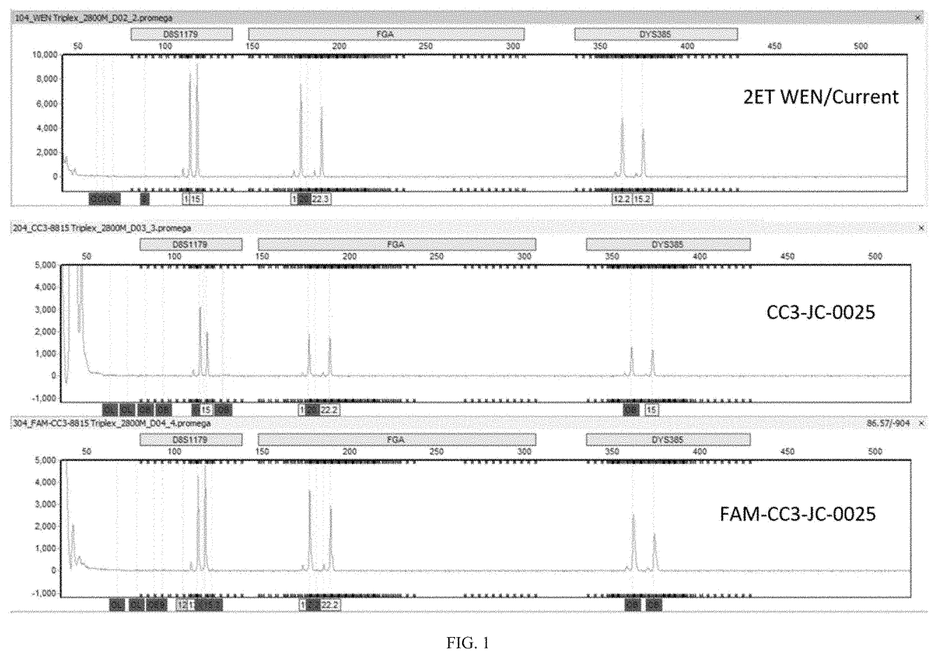

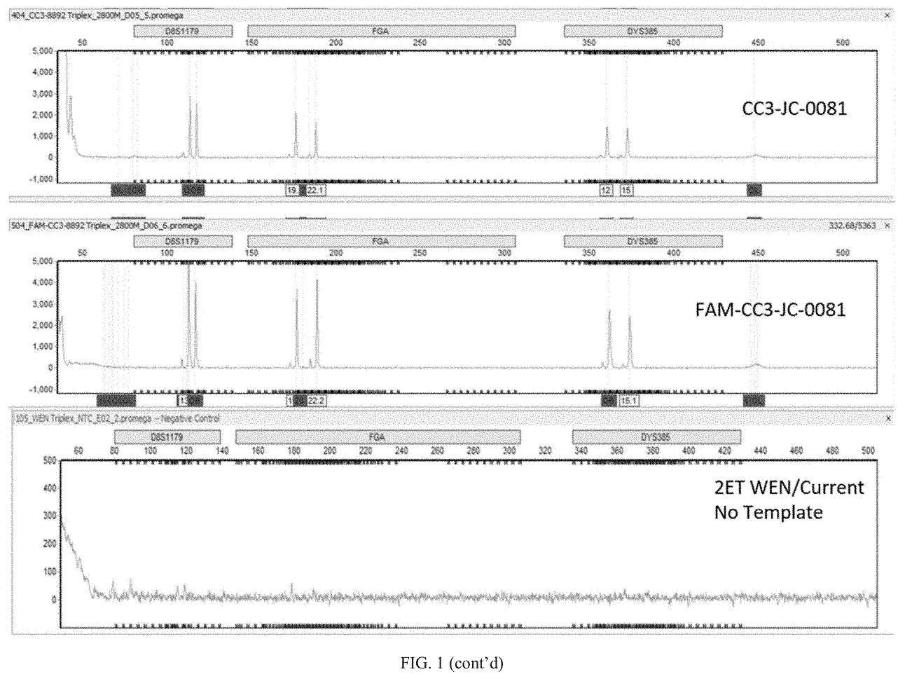

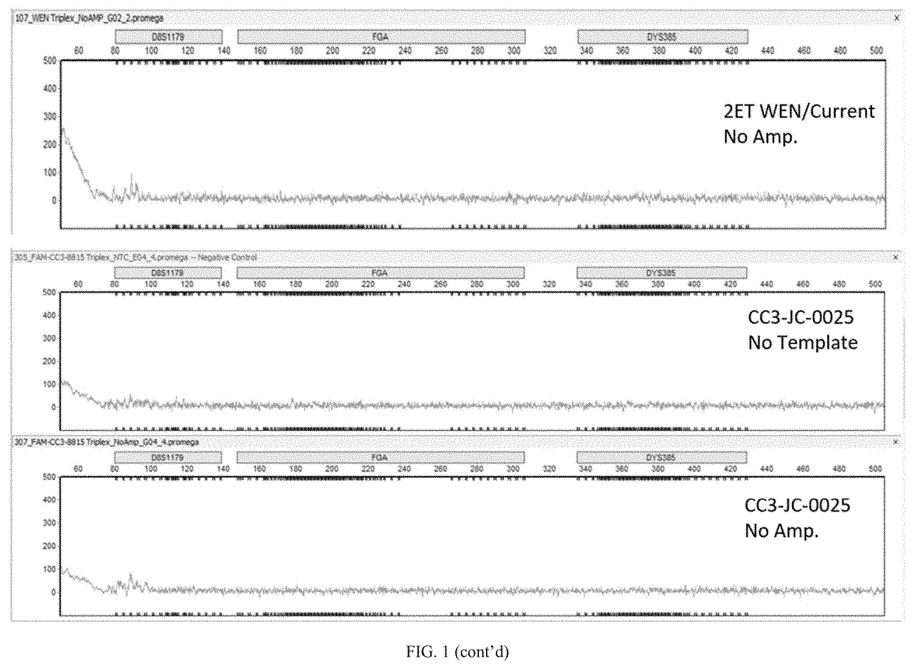

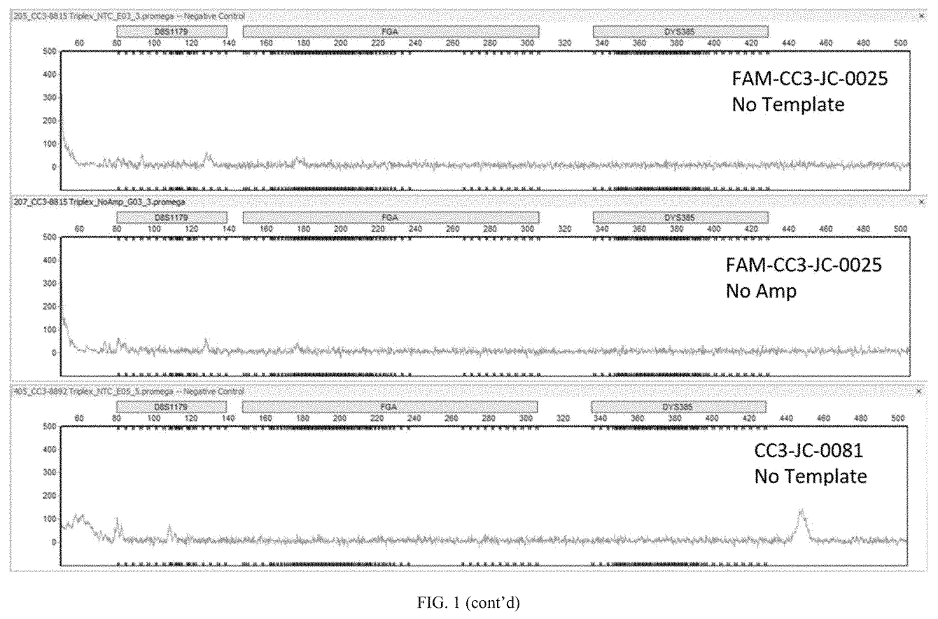

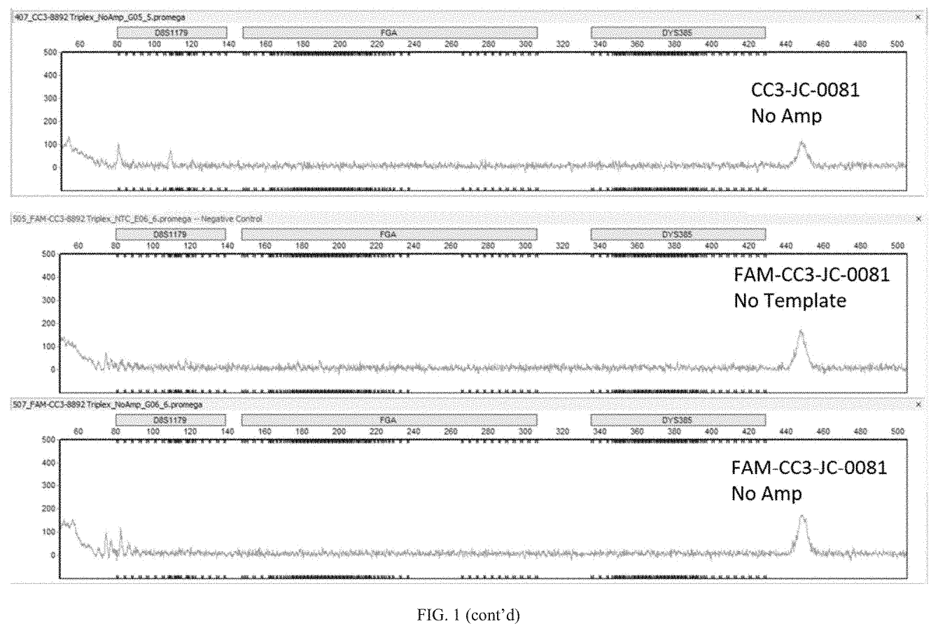

The electropherograms shown in FIG. 1 illustrate that the JC-0025, JC-0081, CS-1341, CS-1377, and JC-0084 dyes were acceptable for use in amplification reactions when compared to the 2ET WEN/Current control.

### Example 4: Multiplex PCR of STRs Using Dyes of the Present Disclosure

Experiments were conducted during development of embodiments of the present invention to determine the energy transfer characteristics of exemplary dyes CS-1341, CS-1377, and JC-0084 for potential use in an 8-dye multiplex PCR of STRs (short tandem repeats).

Oligonucleotides (oligos) were made as described above using the dyes CS-1341, CS-1377, and JC-0084 to derive primer pairs for D8S1179, FGA, and DYS385a/b loci.

The primer pairs were then used in a triplex mix to amplify 2800M DNA. The oligos were then used in amplification reactions that were then analyzed on a Spectrum CE device.

The following amplification reaction conditions were used for each 25 ul reaction:
5× Master Mix: 5 ul
Nanopure water: 12.5 ul
5× Primer Pair (concentrations: D8S1179: 1.0 uM; FGA: 3.20 uM; and DYS385a/b:2.50 uM): 5.0 ul

The reaction mix was vortexed and dispensed into wells of a 96-well plate. 2.5 ul of 2800M DNA was then added to each well. Also included in the reactions: 4 replicates of 2800M and 2 replicate no template (water) controls. The reaction mixes were stored at 4° C. during thermal cycling then run on the CE as “No Amp”.

The reactions were run in an amplification reaction in a ProFlex Thermal cyclers follows:

1 cycle:
96° C. for 1 minute then
29 cycles:
98° C. for 5 seconds
60° C. for 1 min
72° C. for 15 seconds
final extension:
60° C. for 10 minutes
4° C. soak

Amplified samples were then analyzed with a Spectrum CE Beta 02 device. 10 ul of ILS (0.25 CCO ILS 2× Fragment Mix and 9.75 HiDi) were added to each well of a 96-well plate. 1 ul of the amplification reaction or No Amp mix was then added to the appropriate well. The plate was spun briefly to remove bubbles, denatured in a thermal cycler for 3 minutes, and then placed on ice for at least 3 minutes. Samples were injected using a 2 kV, 15 second injection with 8C spectral.

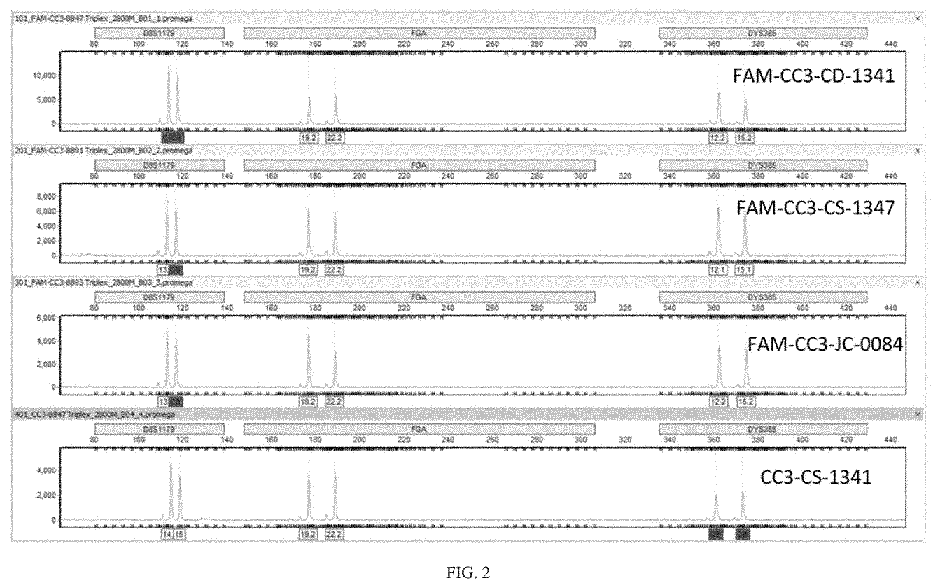

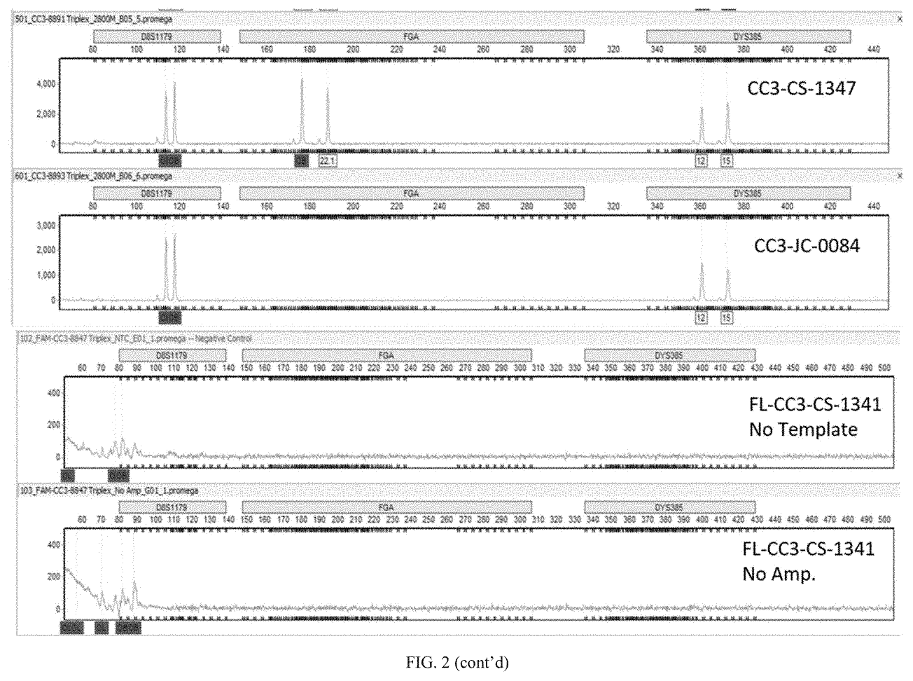

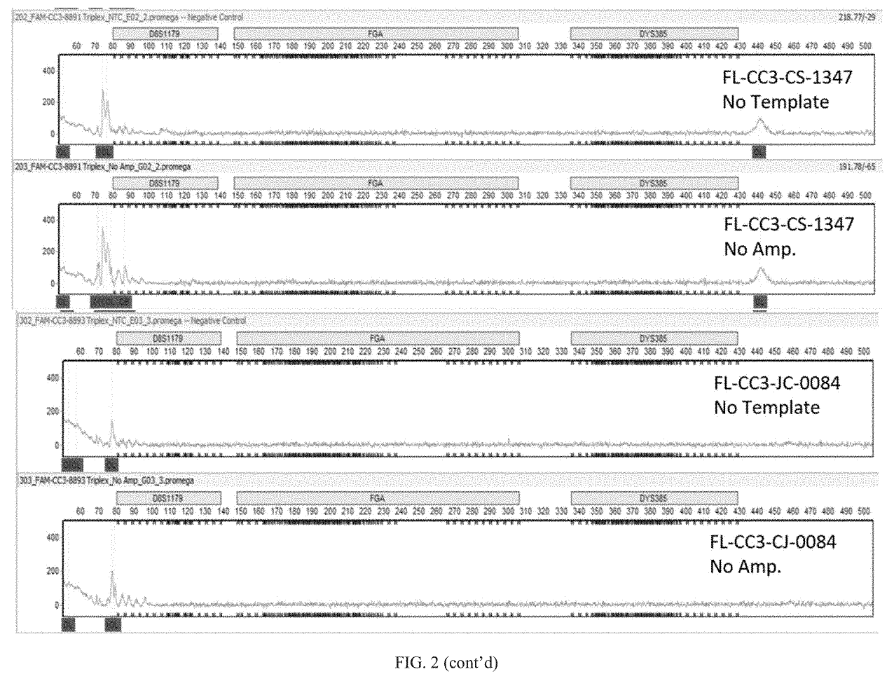

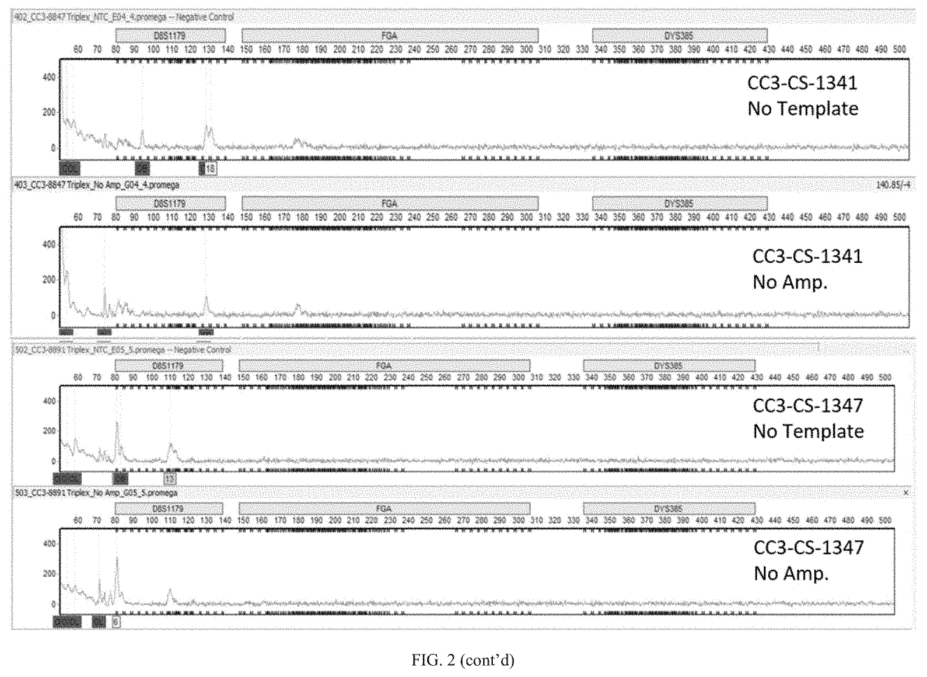

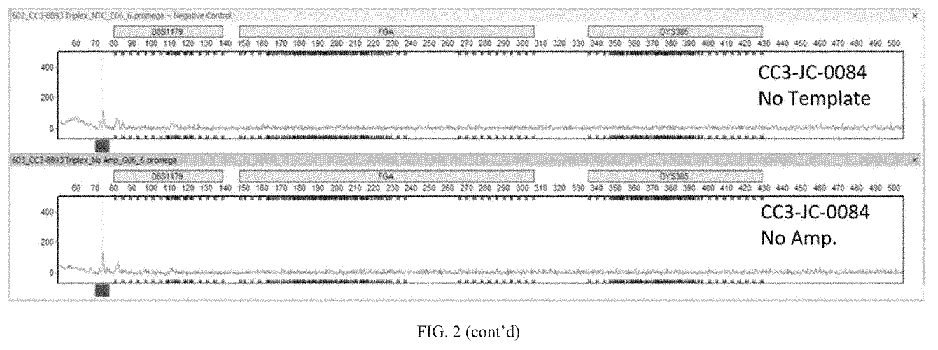

The electropherograms shown in FIG. 2 illustrate that the CS-1341, CS-1377, and JC-0084 dyes were acceptable for use in amplification reactions when compared to the 2ET WEN/Current control.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula (I): 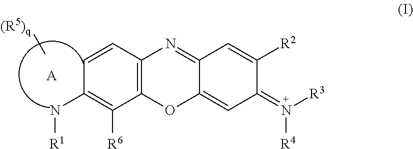
or tautomer or a salt thereof, wherein:
A is a five-, six-, or seven-membered heterocyclyl;
R¹ is selected from hydrogen, C₁-C₆ alkyl, C₁-C₆ heteroalkyl, and -L¹-X, wherein L¹ is alkylene or heteroalkylene, and X is selected from —COOH, —SO₃H, —PO₃H₂, —OPO₃H₂, and a reactive moiety; or R¹ is taken together with R⁶ and the atoms to which they are attached to form a five-, six-, or seven-membered heterocyclyl;
R², R³, and R⁴ are defined follows:
(i) R² is C₁-C₄ alkyl; R³ is hydrogen; and R⁴ is selected from hydrogen, C₁-C₆ alkyl, C₁—C₆ heteroalkyl, and -L²-Z, wherein L² is alkylene or heteroalkylene, and Z is selected from —COOH, —SO₃H, —PO₃H₂, —OPO₃H₂, and a reactive moiety; or
(ii) R² and R³, together with the atoms to which they are attached, form a five-, six-, or seven-membered heterocyclyl; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a reactive moiety; or
(iii) R² is C₁-C₄ alkyl; and R³ and R⁴, together with the nitrogen atom to which they are attached, form a four-, five-, six-, or seven-membered heterocyclyl;
q is 0, 1, 2, or 3; and
each R⁵ is independently selected from C₁-C₄ alkyl; and
R⁶ is hydrogen, or R⁶ is taken together with R¹ and the atoms to which they are attached to form a five-, six-, or seven-membered heterocyclyl;
wherein:
(a) R¹ is -L¹-X, and X is a reactive moiety selected from an active ester, —N₃, —C≡CH, —N═C═O, —N═C═S, maleimido, —C(O)—CH═CH₂, optionally substituted 1,2,4,5-tetrazinyl, cycloalkenyl, and cycloalkynyl; or
(b) R⁴ is -L²-Z, and Z is a reactive moiety selected from an active ester, —N₃, —C≡CH, —N═C═O, —N═C═S, maleimido, —C(O)—CH═CH₂, optionally substituted 1,2,4,5-tetrazinyl, cycloalkenyl, and cycloalkynyl;
and the compound does not have more than one reactive moiety;
wherein the compound is not:

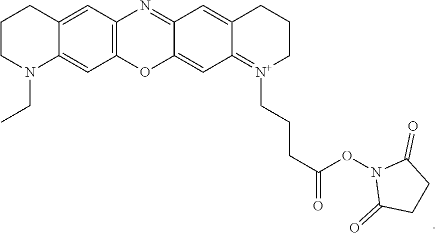 [SMILES: CCN1CCCc2cc3c(cc21)Oc1cc2c(cc1=N3)CCC[N+]=2CCCC(=O)ON1C(=O)CCC1=O]

2. The compound of claim 1, or a tautomer or a salt thereof, wherein A is selected from a pyrrolidine, piperidine, and morpholine ring.

3. The compound of claim 1, or a tautomer or a salt thereof, wherein R¹ is hydrogen, C₁-C₆ alkyl, or C₁-C₆ heteroalkyl.

4. The compound of claim 1, or a tautomer or a salt thereof, wherein R¹ is -L¹-X, wherein L¹ is C₂-C₄ alkylene, and X is selected from —COOH, —SO₃H, —POSH, and a reactive moiety, wherein the reactive moiety is selected from an active ester, —N₃, —C≡CH, —N═C═O, —N═C═S, maleimido, —C(O)—CH═CH₂, tetrazinyl, cycloalkenyl, and cycloalkynyl.

5-6. (canceled)

7. The compound of claim 1, or a tautomer or a salt thereof, wherein R² is C₁-C₄ alkyl; R³ is hydrogen; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a reactive moiety.

8. The compound of claim 1, or a tautomer or a salt thereof, wherein R² and R³, together with the atoms to which they are attached, form a five-, six-, or seven-membered heterocyclyl; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a reactive moiety.

9. The compound of claim 7, or a tautomer or a salt thereof, wherein R⁴ is hydrogen, C₁-C₆ alkyl, or C₁-C₆ heteroalkyl.

10. The compound of claim 7, or a tautomer or a salt thereof, wherein R⁴ is -L²-Z, wherein L² is C₂-C₄ alkylene, and Z is selected from —COOH, —SO₃H, and a reactive moiety, wherein the reactive moiety is selected from an active ester, —N₃, —C≡CH, —N═C═O, —N═C═S, maleimido, —C(O)—CH═CH₂, optionally substituted 1,2,4,5-tetrazinyl, cycloalkenyl, and cycloalkynyl.

11-12. (canceled)

13. The compound of any one of claim 1, or a tautomer or a salt thereof, wherein R² is C₁-C₄ alkyl; and R³ and R⁴, together with the nitrogen atom to which they are attached, form a four-, five-, six-, or seven-membered heterocyclyl.

14. The compound of claim 1, or a tautomer or a salt thereof, wherein q is 0.

15. The compound of claim 1, or a tautomer or a salt thereof, wherein R⁶ is hydrogen.

16. The compound of claim 1, or a tautomer or a salt thereof, wherein:
A is a pyrrolidine ring;
R¹ is -L¹-X, wherein L¹ is C₂-C₄ alkylene and X is an N-succinimidyl ester reactive moiety;
R² and R³, together with the atoms to which they are attached, form a five-membered heterocyclyl having one nitrogen atom;
R⁴ is selected from C₁-C₆ alkyl and -L-Z, wherein L² is C₂-C₄ alkylene and Z is —SO₃H;
q is 0; and
R⁶ is hydrogen.

17. The compound of claim 1, or a tautomer or a salt thereof, wherein:
A is a morpholine ring;
R¹ is -L¹-X, wherein L¹ is C₂-C₄ alkylene and X is an N-succinimidyl ester reactive moiety;
R² is C₁-C₄ alkyl and R³ is hydrogen; or R² and R³, together with the atoms to which they are attached, form a six-membered ring having one nitrogen atom and one oxygen atom;
R⁴ is selected from C₁-C₆ alkyl and -L-Z, wherein L² is C₂-C₄ alkylene and Z is —SO₃H;
q is 0; and
R⁶ is hydrogen.

18. The compound of claim 1, wherein the compound is selected from: 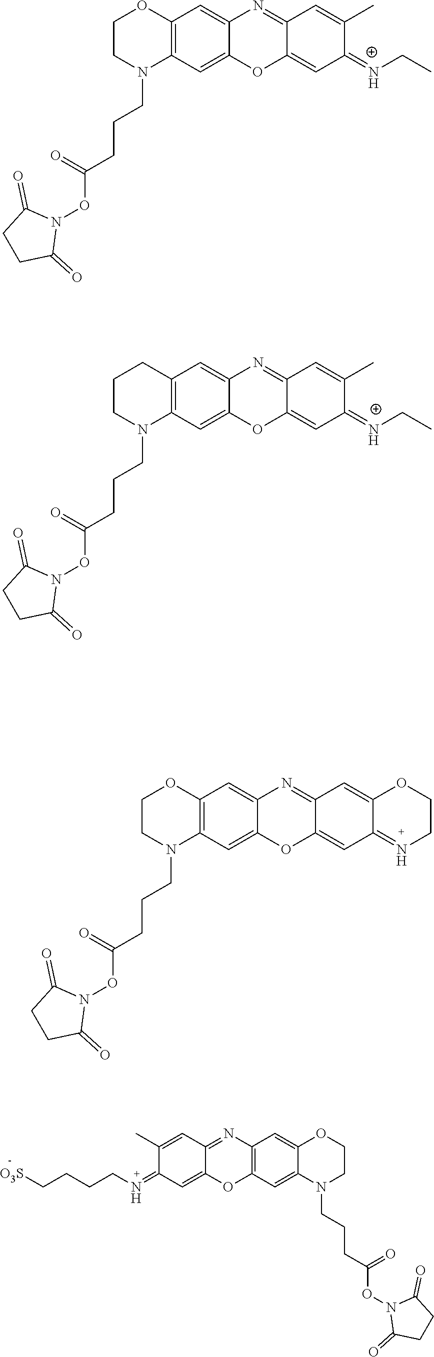 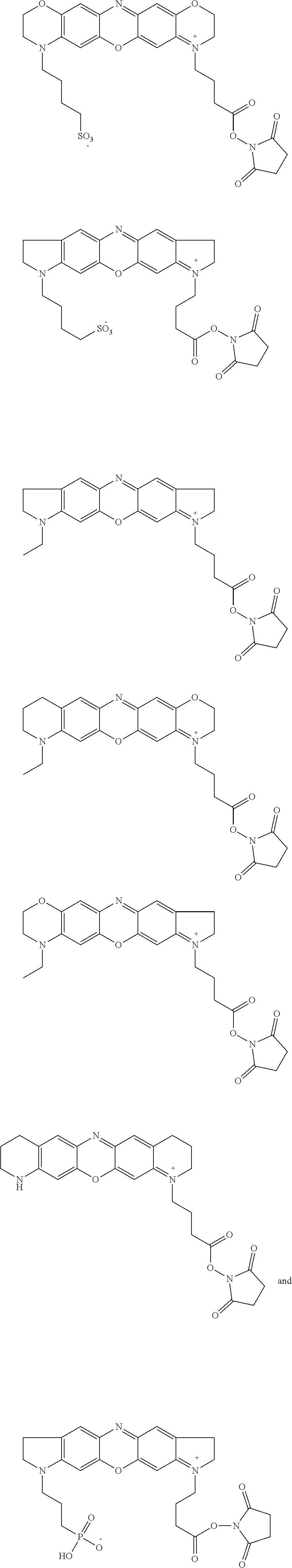
or a tautomer thereof, or a salt thereof.

19. An oligonucleotide comprising a moiety of formula (Ia): 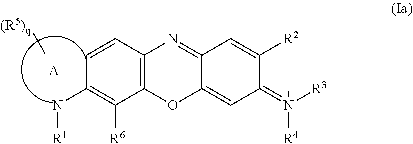
or tautomer or a salt thereof, wherein:
A is a five-, six-, or seven-membered heterocyclyl;
R¹ is selected from hydrogen, C₁-C₆ alkyl, C₁-C₆ heteroalkyl, and -L¹-X, wherein L¹ is alkylene or heteroalkylene, and X is selected from —COOH, —SO₃H, —PO₃H₂, —OPO₃H₂, and a point of attachment to the oligonucleotide; or R¹ is taken together with R⁶ and the atoms to which they are attached to form a five-, six-, or seven-membered heterocyclyl;
R², R³, and R⁴ are defined follows:
(i) R² is C₁-C₄ alkyl; R³ is hydrogen; and R⁴ is selected from hydrogen, C₁-C₆ alkyl, C₁—C₆ heteroalkyl, and -L²-Z, wherein L² is alkylene or heteroalkylene, and Z is selected from —COOH, —SO₃H, —PO₃H₂, —OPO₃H₂, and a point of attachment to the oligonucleotide; or
(ii) R² and R³, together with the atoms to which they are attached, form a five-, six-, or seven-membered heterocyclyl; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a point of attachment to the oligonucleotide; or
(iii) R² is C₁-C₄ alkyl; and R³ and R⁴, together with the nitrogen atom to which they are attached, form a four-, five-, six-, or seven-membered heterocyclyl;
q is 0, 1, 2, or 3; and
each R⁵ is independently selected from C₁-C₄ alkyl; and
R⁶ is hydrogen, or R⁶ is taken together with R¹ and the atoms to which they are attached to form a five-, six-, or seven-membered heterocyclyl;
wherein:
(a) R¹ is -L¹-X, and X is a point of attachment to the oligonucleotide; or
(b) R⁴ is -L²-Z, and Z is a point of attachment to the oligonucleotide
and the moiety of formula (Ia) does not have more than one point of attachment to the oligonucleotide.

20-41. (canceled)

42. A modified nucleotide triphosphate compound comprising a group of formula (Ib): 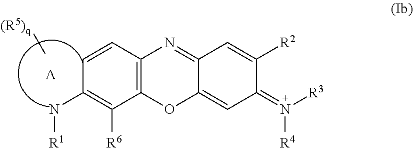
or tautomer or a salt thereof, wherein:
A is a five-, six-, or seven-membered heterocyclyl;
R¹ is selected from hydrogen, C₁-C₆ alkyl, C₁-C₆ heteroalkyl, and -L¹-X, wherein L¹ is alkylene or heteroalkylene, and X is selected from —COOH, —SO₃H, —PO₃H₂, —OPO₃H₂, and a point of attachment to the nucleotide triphosphate compound; or R¹ is taken together with R⁶ and the atoms to which they are attached to form a five-, six-, or seven-membered heterocyclyl;
R², R³, and R⁴ are defined follows:
(i) R² is C₁-C₄ alkyl; R³ is hydrogen; and R⁴ is selected from hydrogen, C₁-C₆ alkyl, C₁—C₆ heteroalkyl, and -L²-Z, wherein L² is alkylene or heteroalkylene, and Z is selected from —COOH, —SO₃H, —PO₃H₂, —OPO₃H₂, and a point of attachment to the nucleotide triphosphate compound; or
(ii) R² and R³, together with the atoms to which they are attached, form a five-, six-, or seven-membered heterocyclyl; and R⁴ is selected from hydrogen, C₁-C₆ alkyl, C₁-C₆ heteroalkyl, and -L²-Z, wherein L² is alkylene or heteroalkylene, and Z is selected from —COOH, —SO₃H, —PO₃H₂, —OPO₃H₂, and a point of attachment to the nucleotide triphosphate compound; or
(iii) R² is C₁-C₄ alkyl; and R³ and R⁴, together with the nitrogen atom to which they are attached, form a four-, five-, six-, or seven-membered heterocyclyl;
q is 0, 1, 2, or 3; and
each R⁵ is independently selected from C₁-C₄ alkyl; and
R⁶ is hydrogen, or R⁶ is taken together with R¹ and the atoms to which they are attached to form a five-, six-, or seven-membered heterocyclyl;
wherein:
(a) R¹ is -L¹-X, and X is a point of attachment to the nucleotide triphosphate compound; or
(b) R⁴ is -L²-Z, and Z is a point of attachment to the nucleotide triphosphate compound,
and the moiety of formula (Ib) does not have more than one point of attachment to the nucleotide triphosphate compound.

43-63. (canceled)

63. The modified nucleotide triphosphate compound of claim 42, wherein the compound is a modified deoxynucleotide triphosphate compound selected from deoxyadenosine triphosphate, deoxycytidine triphosphate, deoxyguanosine triphosphate, and deoxythymidine triphosphate.

64. The modified nucleotide triphosphate compound of claim 42, wherein the compound is a modified dideoxynucleotide triphosphate compound selected from dideoxyadenosine triphosphate, dideoxycytidine triphosphate, dideoxyguanosine triphosphate, and dideoxythymidine triphosphate.

65. A method of performing a nucleic acid amplification reaction, comprising:
(a) adding an oligonucleotide compound of claim 19 to a reaction mixture; and
(b) performing the amplification reaction.

66-67. (canceled)

68. A method of performing a chain termination DNA sequencing reaction, the method comprising:
(a) adding a modified dideoxynucleotide triphosphate compound of claim 64 a polymerase chain reaction (PCR) mixture and performing PCR;
(b) removing unincorporated modified dideoxynucleotide triphosphate compounds from the PCR mixture; and
(c) performing sequencing analysis.

69. (canceled)

70. A method of performing a chain termination DNA sequencing reaction, the method comprising:
(a) adding a modified deoxynucleotide triphosphate compound of claim 63 to a polymerase chain reaction (PCR) mixture, and
(b) performing PCR,
wherein a fluorescent signal from the PCR mixture indicates which dNTP has been added, and wherein a terminator is cleaved to facilitate addition of a subsequent dNTP.

71-72. (canceled)
